(19) United States
(12) Patent Application Publication
(10) Pub. No.: US 2004/0175827 A1
(43) Pub. Date: Sep. 9, 2004

(54) Methods of generating human cd4+ th2 cells and uses thereof

(76) Inventors: Daniel H. Fowler, Bethesda, MD (US); Jeanne Hou, Bethesda, MD (US); Unsu Jung, Ashburn, VA (US); Ronald E. Gress, Gaithersburg, MD (US); Michael Bishop, Rockville, MD (US); Carl June, Merion Station, PA (US); Bruce Levine, Cherry Hill, NJ (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/481,913

(22) PCT Filed: Jun. 26, 2002
(86) PCT No.: PCT/US02/20415

Related U.S. Application Data
(60) Provisional application No. 60/302,936, filed on Jul. 2, 2001.

(51) Int. Cl. A61K 39/395; C12N 5/08

(52) U.S. Cl. 435/372; 424/144.1

(57) ABSTRACT

A method is provided for producing a population of substantially purified CD4⁺ Th2 lymphocytes. The method includes stimulating a population of substantially purified CD4⁺ T cells isolated from a subject by contacting the population with an immobilized anti-CD3 monoclonal antibody and an immobilized antibody that specifically binds to a T cell costimulatory molecule in the presence of a Th2 supportive environment to form a stimulated population of T cells. Purified populations of Th2 cells are disclosed herein, as are methods for their use. For example, substantially purified CD4⁺ Th2 lymphocytes can be used to treat graft-versus-host-disease, tumors, and autoimmune disorders.

### CROSS REFERENCE TO RELATED APPLICATION

This application claims priority to U.S. Provisional Application No. 60/302,936 filed Jul. 2, 2001, herein incorporated by reference in its entirety.

### FIELD

This application relates to the methods for purification of CD4⁺ Th2 cells, to substantially purified populations of CD4⁺ Th2 cells, and to therapeutic uses purified CD4⁺ Th2 cells.

### BACKGROUND

The T lymphocyte (“T cell”) is a key cell type in the human cellular immune system, providing both function and biochemical control. T cells are classified based on which cell surface receptors and cytokines they express. The expression of cell surface receptors CD4 and/or CD8 are generally used to define two broad classes of T cells; these cell surface receptors are involved in recognizing antigens presented to the T cells by antigen presenting cells (APC). Certain mature T cells express only CD4 but not CD8 (termed CD4⁺ cells), while other mature T cells express CD8 but not CD4 (termed CD8⁺ cells).

CD8⁺ cells recognize peptide antigens that are presented on MHC class I molecules. Upon activation by an APC (which involves binding of both a stimulatory antigen and a costimulatory ligand), a CD8⁺ T cell matures into a cytotoxic T cell, which has defined functions and characteristics. CD4⁺ T cells recognize antigens that are presented on MHC class II molecules. When activated by an APC, CD4⁺ T cells can differentiate into T helper (Th) cells. Th cells have been divided into subclasses based on their cytokine secretion profiles. Th1 cells secrete a specific set of cytokines, including interferon-γ (IFN-γ) and interleukin-12 (IL-12), interleukin-2 (IL-2), interferon-γ and lymphotoxin and activate the cellular immunity processes (such as macrophage activation and induction of IgG antibodies by B cells). Th2 cells secrete different cytokines (particularly IL-4, IL-5 and IL-10), and mediate humoral immunity and allergic reactions.

CD4⁺ Th1 and Th2 cells are differentially implicated in immune responses to different diseases and other immune conditions. Recently, techniques have been developed that enable the expansion of mixed populations of T cells in vitro, involving activation of lymphocytes using “artificial APCs” (see, for instance, Garlie et al., 1999; U.S. Pat. No. 5,858,358; and published PCT Application Nos.

US94/06255 and US94/13782). However, obtaining purified populations of CD4⁺ Th1 and Th2 cells separately would be beneficial both for studying the role of these cells, and for treating various disorders.

Donor T cells contained in the blood or marrow allograft mediate both beneficial and detrimental post-transplant immune effects. T cells mediate a potentially curative graft-versus-leukemia (GVL) effect and prevent marrow graft rejection, but also can generate graft-versus-host disease (GVHD). The relative balance of these immune effects is a primary determinant of clinical outcome after allogeneic transplantation. Clinical studies during the 1980's using T cell-depleted (TCD) marrow allografts clearly demonstrated the importance of T cell-mediated immune reactions after alloBMT: recipients of TCD allografts had greatly reduced levels of GVHD, but had much higher rates of both graft rejection and leukemic relapse (Poynton, Bone. Marrow. Transplant. 3:265-79, 1988). Because TCD-alloBMT shifted the cause of mortality from GVHD to leukemia relapse and graft rejection, this approach did not represent a significant treatment advance relative to conventional T cell-replete alloBMT. These observations have prompted investigation into the development of donor T cell administration methodologies which might preserve an anti-leukemic effect and prevent graft rejection while limiting GVHD. Such methods include the administration of only CD4⁺ donor T cells (Champlin et al., Transplant. Proc. 23:1695-6, 1991), or the delayed administration of donor T cells post-transplant (Kolb et al., Blood. 76:2462-5, 1990). Both of these approaches have met with limited success, as leukemia relapse and significant levels of GVHD remain significant problems. Thus, there is a need to purify populations of T cells that can be used to prevent or limit the development of GVHD.

### SUMMARY

Disclosed herein are novel methods for generating CD4⁺ Th2 cells and the purification of these cells. Specifically, culture conditions are disclosed herein that allow Th2 cells to be selectively propagated ex vivo. The ability to grow and administer substantially pure populations of Th2 cells also represents a new therapy to prevent or reduce graft-versus-host disease (GVHD). Thus, the ability to grow Th2 cells represents methods for improving allogeneic stem cell and solid organ transplantation, thus providing treatment for various tumors. In addition, the ability to grow and administer substantially pure populations of Th2 cells also represents a new therapy to treat or alleviate the symptoms of autoimmune disorders in a subject.

In one embodiment, a method is provided for producing a population of substantially purified CD4⁺ Th2 lymphocytes. The method includes stimulating a population of substantially purified CD4⁺ T cells isolated from a subject by contacting the population with an immobilized anti-CD3 monoclonal antibody and an antibody that specifically binds to a T cell costimulatory molecule in the presence of a Th2 supportive environment to form a stimulated population of T cells. In one embodiment, the stimulated population of CD4⁺ T cells is allowed to proliferate in a Th2 supportive environment.

Purified populations of Th2 cells are disclosed herein, as are methods for their use.

The foregoing and other objects, features, and advantages of the methods and cells described herein will become more apparent from the following detailed description of a several embodiments, which proceeds with reference to the accompanying figures.

### BRIEF DESCRIPTION OF THE FIGURES

FIG. 1 is a graph of the T cell yield of human CD4⁺ cells cultured under conditions designed to induce Th1 (lower line) or Th2 (upper line) cell growth. Similar numbers of cells were obtained under the two sets of culture conditions.

FIG. 2 are bar graphs showing the cytokines produced when cells were cultured under conditions designed to generate either Th1 or Th2 cells. The “<” symbol denotes that the cytokine content was below the detection limit for the assay.

FIG. 3 are bar graphs showing the cytokines produced when CD4⁺ cells are further purified into a CD4+RA+ subset or a CD4+RO+ subset, and then cultured under the CD3, CD28 co-stimulation in the specific cytokine conditions that generate Th1 or Th2 cells.

FIG. 4 is an expansion curve which demonstrates that two commonly used immune suppression drugs, cyclosporine A (CSA) and rapamycin (rapa), have different effects on Th2 cell generation.

FIG. 5 are bar graphs showing the cytokines produced when cells were cultured under conditions designed to generate Th2 cells in the presence of CSA or Rapamycin.

FIG. 6 is a flow chart showing the plan for the Phase I/II Study of the use of allogeneic Th2 cells in an allogeneic peripheral blood stem cell transplant.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a cytokine” includes a plurality of such cytokines and reference to “the antibody” includes reference to one or more antibodies and equivalents thereof known to those skilled in the art, and so forth.

Unless explained otherwise, all technical and scientific terms used herein have the same meaning as commonly understood to one of ordinary skill in the art to which this disclosure belongs.

Animal: Living multicellular vertebrate organisms, a category which includes, for example, mammals and birds.

Antibody: Immunoglobulin molecules and immunologically active portions of immunoglobulin molecules, i.e., molecules that contain an antigen binding site which specifically binds (immunoreacts with) an antigen. In one embodiment the antigen is CD3. In another embodiment, the antigen is a co-stimulatory molecule (e.g. CD28).

A naturally occurring antibody (e.g., IgG) includes four polypeptide chains, two heavy (H) chains and two light (L) chains inter-connected by disulfide bonds. However, the antigen-binding function of an antibody can be performed by fragments of a naturally occurring antibody. Thus, these antigen-binding fragments are also intended to be designated by the term antibody. Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-6, 1989) which consists of a VH domain; (v) an isolated complementarity determining region (CDR); and (vi) an F(ab′)₂ fragment, a bivalent fragment comprising two Fab fragments linked by a disulfide bridge at the hinge region. Furthermore, although the two domains of the Fv fragment are coded for by separate genes, a synthetic linker can be made that enables them to be made as a single protein chain (known as single chain Fv (scFv); Bird et al. Science 242:423-6, 1988; and Huston et al., Proc. Natl. Acad. Sci. 85:5879-83, 1988) by recombinant methods. Such single chain antibodies are also included.

In one embodiment, antibody fragments for use in T cell expansion are those which are capable of crosslinking their target antigen, e.g., bivalent fragments such as F(ab′)₂ fragments. Alternatively, an antibody fragment which does not itself crosslink its target antigen (e.g., a Fab fragment) can be used in conjunction with a secondary antibody which serves to crosslink the antibody fragment, thereby crosslinking the target antigen. Antibodies can be fragmented using conventional techniques and the fragments screened for utility in the same manner as described for whole antibodies. An antibody is further intended to include bispecific and chimeric molecules that specifically bind the target antigen.

“Specifically binds” refers to the ability of individual antibodies to specifically immunoreact with an antigen, such as a T cell surface molecule. The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody”.

B Cell: A B cell is a lymphocyte, a type of white blood cell (leukocyte), that develops into a plasma cell, which produces antibodies.

Cancer: Malignant neoplasm that has undergone characteristic anaplasia with loss of differentiation, increase rate of growth, invasion of surrounding tissue, and is capable of metastasis.

Chemotherapy: In cancer treatment, chemotherapy refers to the administration of one or a combination of compounds to kill or slow the reproduction of rapidly multiplying cells. Chemotherapuetic agents include those known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol.

Chemotherapy-resistant disease: A disorder that is not responsive to administration of a chemotherapeutic agent.

Comprises: A term that means “including.” For example, “comprising A or B” means including A or B, or both A and B, unless clearly indicated otherwise.

Costimulator of a T cell: Although stimulation of the TCR/CD3 complex (or CD2 molecule) is required for delivery of a primary activation signal in a T cell, a number of molecules on the surface of T cells, termed accessory or costimulatory molecules have been implicated in regulating the transition of a resting T cell to blast transformation, and subsequent proliferation and differentiation (T cell stimulation). Thus, in addition to the primary activation signal provided through the TCR/CD3 complex, induction of T cell responses requires a second, costimulatory signal. A costimulator of a T cell includes, but is not limited to CD28, inducible costimulatory molecule (ICOS), 4-1BB receptor (CDw137), lymphocyte function-associated antigen-1 (LFA-1), CD30, or CD154.

One such costimulatory or accessory molecule, CD28, is understood to initiate or regulate a signal transduction pathway that is distinct from those stimulated by the TCR complex. Other specific, non-limiting examples of co-stimulatory molecules are inducible costimulatory molecule (ICOS), 4-1BB receptor (CDw137), lymphocyte function-associated antigen-1 (LFA-1), CD30, or CD154 (see Salomon and Bluestone, Ann. Rev. Immunol 19:225-52, 2001).

Thus, to induce an activated population of T cells to proliferate (i.e., a population of T cells that has received a primary activation signal) an accessory molecule on the surface of the T cell (e.g. CD28), is stimulated with a ligand which binds the accessory molecule. In one embodiment, stimulation of the accessory molecule is accomplished by contacting an activated population of T cells with a ligand that binds to the accessory molecule, or with an antibody that specifically binds the accessory molecule.

In one embodiment, activation of CD4⁺ T cells with an anti-CD3 antibody and an anti-CD28 antibody results in selective proliferation of CD4⁺ T cells. An anti-CD28 monoclonal antibody or fragment thereof capable of cross-linking the CD28 molecule, or a natural ligand for CD28 (e.g., a member of the B7 family of proteins, such as B7-1(CD80) and B7-2 (CD86) (Freedman et al., J. Immunol. 137:3260-7, 1987; Freeman et al., J. Immunol 143:2714-22, 1989; Freeman. et al., J. Exp. Med. 174:625-31, 1991; Freeman et al., Science 262:909-11, 1993; Azuma et al., Nature 366:76-9, 1993; Freeman et al., J. Exp. Med. 178:2185-92, 1993) can be used to induce stimulation of the CD28 molecule. In addition, binding homologues of a natural ligand, whether native or synthesized by chemical or recombinant technique, can also be used. Ligands useful for stimulating an accessory molecule can be used in soluble form or immobilized on a solid phase surface as described herein. Anti-CD28 antibodies of fragments thereof useful in stimulating proliferation of CD4⁺ T cells include monoclonal antibody 9.3, an IgG2a antibody (Dr. Jeffery Ledbetter, Bristol Myers Squibb Corporation, Seattle, Wash.), monoclonal antibody KOLT-2, an IgG1 antibody, 15E8, an IgG1 antibody, 248.23.2, an IgM antibody and EX5.3D10, an IgG2a antibody (see U.S. Pat. No. 5,858,358).

Cytokine/Interleukin (IL): A generic name for a diverse group of soluble proteins and peptides which act as humoral regulators at nano—to picomolar concentrations and which, either under normal or pathological conditions, modulate the functional activities of individual cells and tissues. These proteins also mediate interactions between cells directly and regulate processes taking place in the extracellular environment. Many growth factors and cytokines act as cellular survival factors by preventing programmed cell death. Cytokines and interleukins include both naturally occurring peptides and variants that retain fill or partial biological activity. Although specific cytokines/interleukins are described in the specification, they are not limited to the specifically disclosed peptides.

Enhance: To improve the quality, amount, or strength of something. In one embodiment, a therapy enhances the ability of a subject to reduce GVHD, an autoimmune disorder, and/or tumors in the subject if the subject is more effective at fighting GVHD, an autoimmune disorder, and/or tumors. Such enhancement can be measured using the methods disclosed herein, for example determining the level of type II cytokines produced using an ELISA, or determining the decrease in GVHD, an autoimmune disorder, and/or tumor.

Immobilized: Bound to a surface, such as a solid surface. A solid surface can be polymeric, such as polystyrene or polypropylene. In one embodiment, the solid surface is the bottom surface of a flask or a tissue culture plate. In another embodiment, the solid surface is in the form of a bead A specific, non-limiting example of a bead is Tosylated magnetic beads (Dynal). Methods of immobilizing antibodies and peptides on a solid surface can be found in WO 94/29436, and U.S. Pat. No. 5,858,358.

Immuno-deplete: To decrease the number of lymphocytes, such as CD4⁺ and/or CD8⁺ cells, in a subject.

Immuno-depleting agent: One or more compounds, when administered to a subject, result in a decrease in the number of cells of the immune system (such as lymphocytes) in the subject. Examples include, but are not limited to, chemotherapeutic agents, monoclonal antibodies, and other therapies disclosed in EXAMPLE 8.

Immunologically Normal: A subject that displays immune system characteristics typical for the species to which the subject belongs. These characteristics would typically include, among others, functioning B-cells and T-cells as well as structural cell components, called cell surface antigens, which act as the immunologic signature for a particular organism.

The use of such immunologically normal recipients means that an immunologically normal recipient's immune system, via its B-(humoral response) and T-(cellular response) cells, will identify the cell surface antigens of a foreign cell or an engrafted tissue as foreign. This recognition leads ultimately to an immune response against the cell or tissue, resulting in destruction of the cell or rejection of the graft. An immune response against an allogeneic tissue is known as host-versus-graft rejection. The graft can be a solid organ, such as a heart, kidney, liver, or pancreas.

Immunologically Compromised: An “immunologically compromised recipient” is a subject with a genotypic or a phenotypic immunodeficiency.

A genotypically-immunodeficient subject has a genetic defect which result in the inability to generate either humoral or cell-mediated response. A specific, non-limiting example of a genotypically immunodeficient subject is a genotypically immunodeficient mouse, such as a SCID mouse or a bg/nu/xid mice (Andriole et al., J. Immunol. 135:2911, 1985; McCune et al., Science 241:1632, 1988). In one example, a genotypically immunodeficient subject is unable to react against a foreign cell or engrafted allogeneic tissue.

A phenotypically-immunodeficient subject is genetically capable of generating an immune response, yet has been phenotypically altered such that no response is seen. In one specific, non-limiting example, a phenotypically-immunodeficient recipient is irradiated. In another specific, non-limiting example, a phenotypically-immunodeficient subject has been treated with chemotherapy.

Interferon-gamma (IFN-γ): A dimeric protein glycosylated at two sites with subunits of 146 amino acids. Murine and human IFN-γ have approximately 40% sequence homology at the protein level. The human IFN-γ gene is approximately 6 kb, contains four exons and maps to chromosome 12q24.1. At least six different variants of naturally occurring IFN-γ have been described, and differ from each other by variable lengths of the C-terminal ends. IFN-γ includes both naturally occurring peptides, as well as IFN-γ fragments and variants that retain full or partial biological activity.

In T helper cells (Th cells) IL2 induces the synthesis of IFN-γ and other cytokines. IFN-γ also stimulates the expression of Ia antigens on the cell surface, the expression of CD4 in T helper cells, and the expression of high-affinity receptors for IgG in myeloid cell lines, neutrophils, and monocytes.

IFN-γ can be detected by immunoassay. A specific ELISA test allows detection of individual cells producing IFN-γ. Minute amounts of IFN-γ can be detected indirectly by measuring IFN-induced proteins such as Mx protein. The induction of the synthesis of IP-10 has been used also to measure IFN-γ concentrations. A new bioassay employs induction of indoleamine 2,3-dioxygenase activity in 2D9 cells. A sensitive radioreceptor assay is also available.

IL-2: A protein of 133 amino acids (15.4 kDa) with a slightly basic pI. IL-2 does not display sequence homology to any other factors. Murine and human IL-2 display a homology of approximately 65 percent. IL2 is synthesized as a precursor protein of 153 amino acids with the first 20 amino terminal amino acids functioning as a hydrophobic secretory signal sequence. The protein contains a single disulfide bond (positions Cys58/105) essential for biological activity. Naturally occurring IL-2 is O-glycosylated at threonine at position 3. However, variants exist with different molecular masses and charges are due to variable glycosylation. Non-glycosylated IL-2 is also biologically active. Glycosylation appears to promote elimination of the factor by hepatocytes. It is understood that IL-2 includes both naturally occurring and recombinant IL-2 peptides, as well as IL-2 fragments and IL-2 variants that retain full or partial IL-2 biological activity.

The human IL-2 gene contains four exons. The IL-2 gene maps to human chromosome 4q26-28, while the mouse gene maps to murine chromosome 3. The homology of murine and human IL-2 is 72 percent at the nucleotide level in the coding region.

Mouse and human IL-2 both cause proliferation of T-cells of the homologous species at high efficiency. Human IL-2 also stimulates proliferation of mouse T-cells at similar concentrations, whereas mouse IL-2 stimulates human T-cells at a lower (sixfold to 170-fold) efficiency. IL-2 is a growth factor for all subpopulations of T-lymphocytes. It is an antigen-unspecific proliferation factor for T-cells that induces cell cycle progression in resting cells, and allows clonal expansion of activated T-lymphocytes. Due to its effects on T-cells and B-cells IL-2 is considered to be a central regulator of immune responses (Waguespack et al., Brain. Research Bull. 34: 103-9, 1994)

IL-2 can be assayed in bioassays employing cell lines that respond to the factor (e.g., ATH8, CT6, CTLL-2, FDCPmix, HT-2, NKC-3, TALL-103). Specific ELISA assays for IL-2 and enzyme immunoassays for the soluble receptor are also available. An alternative detection method is reverse transcriptase polymerase chain reaction (RT-PCR) (e.g. see Brandt et al., Lymphokine Research 5:S35-S42 1986; Lindquist et al., J. Immunol. Meth. 113:231-5 1988).

IL-4: IL-4 is a protein produced mainly by a subpopulation of activated T-cells (CD4⁺TH2 cells), which also secrete IL-5 and IL-6. IL-4 is 129 amino acids (20 kDa) that is synthesized as a precursor containing a hydrophobic secretory signal sequence of 24 amino acids. IL-4 is glycosylated at two arginine residues (positions 38 and 105) and contains six cysteine residues involved,in disulfide bond formation. Some glycosylation variants of IL-4 have been described that differ in their biological activities. A comparison of murine and human IL-4 shows that both proteins only diverge at positions 91-128. It is understood that IL-4 includes both naturally occurring and recombinant IL-4 peptides, as well as IL-4 fragments and IL-4 variants that retain full or partial IL-4 biological activity.

The human IL-4 gene contains four exons and has a length of approximately 10 kb. It maps to chromosome 5q23-31, while the murine gene maps to chromosome 11. At the nucleotide level the human and the murine IL-4 gene display approximately 70 percent homology.

The biological activities of IL-4 are species-specific; mouse IL-4 is inactive on human cells and human IL-4 is inactive on murine cells. IL-4 promotes the proliferation and differentiation of activated B-cells, the expression of class II MHC antigens, and of low affinity IgE receptors in resting B-cells. In addition, IL-4 is known to enhance expression of class II MHC antigens on B-cells. This cytokine also can promote the B-cells' capacity to respond to other B-cell stimuli and to present antigens for T-cells.

The classical detection method for IL-4 is a B-cell costimulation assay measuring the enhanced proliferation of stimulated purified B-cells. IL-4 can be detected also in bioassays, employing IL4-responsive cells (e.g. BALM-4, BCL1, CCL-185, CT.4S, amongst others). A specific detection method for human IL-4 is the induction of CD3 in a number of B-cell lines with CD23 detected either by flow-through cytometry or by a fluorescence immunoassay. An alternative and entirely different detection method is RT-PCR (for review see: Boulay and Paul, Current Opinion in Immunology 4:294-8, 1992; Paul and Ohara, Annual Review of Immunology 5:429-59, 1987).

IL-5: Murine IL-5 cDNA encodes a protein of 113 amino acids, while the human protein is 115 amino acids. Murine and human IL-5 protein sequences are approximately 70% identical. The biologically active form of IL-5 is an N-glycosylated antiparallel homodimer linked by disulfide bonds. Monomeric forms are biologically inactive. Non-glycosylated IL-5 is also biologically active. However, it is understood that IL-5 includes both naturally occurring and recombinant IL-5 peptides, as well as IL-5 fragments and IL-5 variants that retain full or partial IL-5 biological activity.

IL-5 promotes the generation of cytotoxic T-cells from thymocytes. In thymocytes, IL-5 induces the expression of high affinity IL-2 receptors.

IL-10: A homodimeric protein with subunits having 160 amino acids. Human IL-10 shows 73% amino acid homology with murine IL-10, and 81% homology with murine IL-10 at the nucleotide level. However, it is understood that IL-10 includes both naturally occurring and recombinant IL-10 peptides, as well as IL-10 fragments and IL-10 variants that retain full or partial IL-10 biological activity.

IL-10 is produced, for example, by activated CD8+ peripheral blood T-cells and by Tc2 cells.

IL-10 can inhibit the synthesis of a number of cytokines such as IFN-γ, IL-2 and TNF-β in Tc1 subpopulations of T-cells. This activity can be antagonized by IL-4. IL-10 also inhibits mitogen- or anti-CD3-induced proliferation of T-cells in the presence of accessory cells and reduces the production of IFN-γ and IL-2.

Several methods can be used to detect IL-10, including, but not limited to: ELISA; using the murine mast cell line D36 can be used to bioassay human IL-10; and flow cytometry.

IL-12: IL-12 is a heterodimeric 70 kDa glycoprotein consisting of a 40 kDa subunit and a 35 kDa subunit linked by disulfide bonds. However, it is understood that IL-12 includes both naturally occurring and recombinant IL-12 peptides, as well as IL-12 fragments and IL-12 variants that retain full or partial IL-12 biological activity.

IL-12 is secreted by peripheral lymphocytes after induction. It is produced mainly by B-cells and to a lesser extent by T-cells. The most powerful inducers of IL-12 are bacteria, bacterial products, and parasites. IL-12 is produced after stimulation with phorbol esters or calcium ionophore by human B-lymphoblastoid cells. IL-12 activates NK-cells positive for CD56, and this activity is blocked by antibodies specific for TNF-alpha.

IL-12 can be detected by assaying its activity as a NKSF (natural killer cell stimulatory factor), by a CLMF (cytotoxic lymphocyte maturation factor), flow cytometry, ELISA, or RT-PCR using standard methodologies and as described herein.

Isolated: An “isolated” biological component (such as a nucleic acid molecule, protein, vascular tissue or hematological material, such as blood components) has been substantially separated or purified away from other biological components in the cell of the organism in which the component naturally occurs. Vascular tissue that has been isolated includes separation by surgical and/or enzymatic methods. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

An isolated cell, is one which has been substantially separated or purified away from other biological components of the organism in which the cell naturally occurs. For example, an isolated CD4⁺ cell population is a population of CD4⁺ cells which is substantially separated or purified away from other blood cells, such as CD8⁺ cells. An isolated Th2 cell population is a population of Th2 cells which is substantially separated or purified away from other blood cells, such as Th1 cells.

Lymphocytes: A type of white blood cell that is involved in the immune defenses of the body. There are two main types of lymphocytes: B-cells and T-cells.

Lymphoproliferation: An increase in the production of lymphocytes.

Malignant: Cells which have the properties of anaplasia invasion and metastasis.

Mammal: This term includes both human and non-human mammals. Similarly, the term “subject” includes both human and veterinary subjects.

Monocyte: A large white blood cell in the blood that ingests microbes or other cells and foreign particles. When a monocyte passes out of the bloodstream and enters tissues, it develops into a macrophage.

Neoplasm: Abnormal growth of cells.

Neutralizing amount: An amount of an agent sufficient to decrease the activity or amount of a substance to a level that is undetectable using standard method.

Non-cultured Cells: Cells which have not been grown or expanded outside of the body. In one embodiment, non-cultured CD4⁺ and CD8⁺ T cells are cells that have been removed and purified from the body, but not grown in culture.

Normal Cell: Non-tumor cell, non-malignant, uninfected cell.

Pharmaceutically acceptable carriers: The pharmaceutically acceptable carriers useful with the methods described herein are conventional. Remington's Pharmaceutical Sciences, by E. W. Martin, Mack Publishing Co., Easton, Pa., 15th Edition (1975), describes compositions and formulations suitable for pharmaceutical delivery of the cytokines and cells disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Purified: The term “purified” does not require absolute purity; rather, it is intended as a relative term. Thus, for example, a substantially purified protein preparation is one in which the protein referred to is more pure than the protein in its natural environment within a cell or within a production reaction chamber (as appropriate). Substantially purified populations of cells refers to populations of cells that are at least 80%, 90%, 95%, 96%, 97%, 98% or 99% pure. In one ernrbodiment, a substantially purified population of Th2 cells is composed of about 95% Th2 cells, that is the population of cells includes less than about 5% of other T lymphocytes such as Th1 cells. The purity of a Th2 population can be measured based on cell surface characteristics (e.g. as measured by fluorescence activated cell sorting) or by cytokine secretion profile, as compared to a control.

Thus, in one example, a substantially purified population of CD4⁺ T cells demonstrates a 95% reduction in IL-2 secretion relative to a control Th1 population from the same donor. In another embodiment, a population of substantially purified Th2 cells is about 99% Th2 cells, that is the population of cells includes less than about 1% of other T lymphocytes such as Th1 cells. In one specific, non-limiting example, a substantially purified population of CD4⁺ T cells demonstrates a 99% reduction in IL-2 secretion relative to a control CD4⁺Th1 population from the same donor.

One specific, non-limiting example of a purified population of CD4⁺ Th2 cells is a CD4⁺ population of cells that produces less than 200 pg/ml of IL-2 per 1×10⁶ CD4⁺ Th2 lymphocytes, for example less than 100 pg/ml of IL-2 per 1×10⁶ CD4⁺ Th2 lymphocytes, for example less than 10 pg/ml of IL-2 per 1×10⁶ CD4⁺ Th2 lymphocytes.

In further embodiments, a substantially purified population of Th2 cells is a CD4⁺ population of cells that produces at least 200 pg/ml of IL-4 per 1×10⁶ CD4⁺ Th1 lymphocytes, for example at least 500 pg/ml of IL-4 per 1×10⁶ CD4⁺ Th1 lymphocytes, for example at least 1000 pg/ml of IL-4 per 1×10⁶ CD4⁺ Th2 lymphocytes.

Stem Cell: A pluripotent cell that gives rise to progeny in all defined hematolymphoid lineages. In addition, limiting numbers of cells are capable of fully reconstituting a seriously immunocompromnised subject in all blood cell types and their progenitors, including the pluripotent hematopoietic stem cell by cell renewal.

Subject: Any subject that has a vascular system and has hematopoietic cells in the wild-type organism. The methods disclosed herein have equal application in medical and veterinary settings. Therefore, the general term “subject being treated” is understood to include all organisms (e.g. humans, apes, dogs, cats, mice, rats, rabbits, horses, pigs, and cows) that require an increase in the desired biological effect.

Substantially Free: Below the limit of detection for a given assay. Thus, in one specific non-limiting example, a cell culture is substantially free of IL-2 if it cannot be detected by a standard assay for analyzing IL-2 expression (e.g. below 10 pg/ml IL-2). In one example, the assay is a bioassay or an ELISA assay for a specific cytokine, wherein appropriate controls are utilized to document the absence of expression of the cytokine.

Supernatant: The culture medium in which a cell is grown. The culture medium includes material from the cell, including secreted growth factors.

Therapeutically Effective Amount: An amount sufficient to achieve a desired biological effect, for example an amount that is effective to decrease the effects and/or severity of GVHD, for example after allogeneic bone marrow transplantation or solid organ transplantation. In one example, it is an amount sufficient to increase a graft-versus-leukemia (GVL) or graft-versus-tumor (GVT) effect. In yet another example, it is an amount sufficient to decrease the symptoms or effects of a tumor, such as a carcinoma or a hematologic or lymphoid malignancy. In yet another example, it is an amount sufficient to decrease the symptoms or effects of an autoimmune disorder. In particular examples, it is an amount of Th2 cells effective to decrease the effects of GVHD, such as in a subject to whom it is administered, such as a subject having one or more tumors. In other examples, it is an amount of Th2 cells effective to decrease the effects of an autoimmune disorder.

In one embodiment, the therapeutically effective amount also includes a quantity of purified Th2 cells sufficient to achieve a desired effect in a subject being-treated. For instance, these can be an amount necessary to improve signs and/or symptoms a disease such as GVHD, and autoimmune disorder and/or cancer.

An effective amount of purified Th2 cells can be administered in a single dose, or in several doses, for example daily, during a course of treatment However, the effective amount of purified Th2 cells will be dependent on the subject being treated, the severity and type of the condition being treated, and the manner of administration. For example, a therapeutically effective amount of purified Th2 cells can vary from about 5×10⁶ cells per kg body weight to about 1.25×10⁸ cells per kg body weight, for example about 25×10⁶ cells per kg body weight.

Therapeutically effective dose: In one example, a dose of purified Th2 cells sufficient to decrease GVHD in a subject to whom it is administered, resulting in a regression of a pathological condition, or which is capable of relieving signs or symptoms caused by the condition. In a particular example, it is a dose of purified Th2 cells sufficient to decrease a GVHD response in a subject after an allogeneic bone marrow transplant or a solid organ transplant. In yet another embodiment, it is a dose of purified Th2 cells sufficient to improve a graft-versus-leukemia (GVL) effect in a subject. In another example, it is an amount sufficient to decrease the symptoms or effects of a tumor, such as a carcinoma or a hematologic or lymphoid malignancy.

In another example, it is a dose of purified Th2 cells sufficient to decrease the effects of an autoimmune disorder in a subject to whom it is administered, resulting in a regression of a pathological condition, or which is capable of relieving signs or symptoms caused by the condition.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte. In another embodiment, a CD8⁺ cell is a suppressor T cell.

T cell stimulation: A state in which a T cell response has been initiated or activated by a primary signal, such as through the TCR/CD3 complex, but not necessarily due to interaction with a protein antigen. T cell stimulation includes stimulation of a T cell with a primary signal (e.g. anti-CD3) and a co-stimulatory molecule (e.g. anti-CD28). A T cell is activated if it has received a primary signaling event that initiates an immune response by the T cell.

T cell stimulation can be accomplished by stimulating the T cell TCR/CD3 complex or via stimulation of the CD2 surface protein. An anti-CD3 monoclonal antibody can be used to activate a population of T cells via the TCR/CD3 complex. A number of anti-human CD3 monoclonal antibodies are commercially available. For example, OKT3 prepared from hybridoma cells obtained from the American Type Culture Collection and monoclonal antibody G19-4 can be used to activate T cells. Similarly, binding of an anti-CD2 antibody will activate T cells.

Th1 and Th2 Cells: Type-1 helper cells (Th1), but not type-2 helper cells (Th2), are CD4⁺ T cells that secrete Th1 cytokines. Specific, non-limiting examples of Th1 cytokines are IL-2, interferon gamma (IFN-γ), and tumor necrosis factor beta (TNF-β). Th2 cells, but not Th1 cells, express Th2 cytokines. Specific, non-limiting examples of Th2 cytokines are IL-4, IL-5, IL-6, and IL-10.

The different patterns of cytokine secretion have been postulated correspond with different functions as immune effectors. Th1 cells are known to promote cell-mediated effector responses, while Th2 cells are helper cells that influence B-cell development and augment humoral responses such as the secretion of antibodies, predominantly of IgE, by B-cells. Both types of Th cells influence each other by the cytokines they secrete. For example, IFN-γ can inhibit the proliferation of murine Th2 cells but not that of Th1 helper T-lymphocyte clones. In contrast, IL-10 from Th2 cells can inhibit the proliferation of Th1 cells. Multiple murine models, including infectious disease, cancer, transplantation, and autoimmune models, have demonstrated that such a Th1/Th2 immune balance contributes significantly to the natural history of these various conditions.

Tumor: A neoplasm. Includes solid and hematological (or liquid) tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (including low-, intermediate-, and high-grade), multiple myeloma, Waldenstrdm's macroglobulinemia, heavy chain disease, myelodysplastic syndrome, mantle cell lymphoma and myelodysplasia.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wihms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Transplantation: The transfer of a tissue, cells, or an organ, or a portion thereof, from one body or part of the body to another body or part of the body. An “allogeneic transplantation” or a “heterologous transplantation” is transplantation from one individual to another, wherein the individuals have genes at one or more loci that are not identical in sequence in the two individuals. An allogeneic transplantation can occur between two individuals of the same species, who differ genetically, or between individuals of two different species. An “autologous transplantation” is a transplantation of a tissue, cells, or a portion thereof from one location to another in the same individual, or transplantation of a tissue or a portion thereof from one individual to another, wherein the two individuals are genetically identical.

### Method for Purifying and Expanding CD4⁺ Th2 Cells

A method of producing a population of substantially purified CD4⁺ Th2 lymphocytes is provided herein The method includes isolating or obtaining CD4⁺ cells from a subject In one example the subject is an HLA-matched donor. In another example, the subject has at least one tumor, such as a solid or hematological tumor. In one embodiment, the method includes further purifying a CD4⁺RO⁺ T cell subset of CD4⁺ cells.

In one embodiment, CD4⁺ T cells are isolated via cell sorting. One specific, non-limiting example of a method of isolating CD4⁺ cells is the use of negative magnetic immunoadherence. This method uses a cocktail of monoclonal antibodies directed to cell surface markers present on the cells negatively selected. For example, to isolate cells, a monoclonal antibody cocktail may include antibodies to CD14 (e.g. monoclonal antibody 63D3, or 20.3), CD20 (e.g. monoclonal antibody IF5 or Leu-16), CD11b (monoclonal antibody OKMI or 60.1), CD16 (monoclonal antibody FC-2.2 or 3G8), HLA-DR (e.g. monoclonal antibody 20.6 or HB10a) and CD8 (e.g. monoclonal antibody OKT8, 51.1, or G10-1.1). This process of negative selection results in an essentially homogenous population of CD4⁺ cells (see U.S. Pat. No. 5,858,358). However, this method is exemplary, other methods known to those of skill in the art can also be utilized.

In another embodiment, purified populations of CD4⁺RO⁺ T cells are isolated via cell sorting. One specific, non-limiting example of a method of isolating CD4⁺RO⁺ T cells is the use of positive selection. Using antibodies directed to the RO antigen on CD4 cells to mark the RO subset of CD4 cells, the CD4⁺RO⁺ T cells can be purified by flow sorting.

The purified CD4⁺ T cells are stimulated by contacting the cells with an immobilized anti-CD3 antibody and an immobilized antibody that specifically binds to a T cell costimulatory molecule. In one example, the antibodies are immobilized. In a particular example, the antibodies are immobilized on a bead, a magnetic solid phase surface, or adhered to a tissue culture flask. T cell costimulatory molecules include, but are not limited to, CD28, inducible costimulatory molecule (ICOS), 4-1BB receptor (CDw137), lymphocyte function-associated antigen-1 (LFA-1), CD30, or CD154. Methods of stimulation of T cells with immobilized anti-CD3 and an immobilized costimulatory molecule are known (see U.S. Pat. No. 3,858,350 and PCT WO 94/29436, herein incorporated by reference in their entirety). The CD4⁺ T cells can be stimulated once. In another example, the population of T cells is re-stimulated with the immobilized anti-CD3 and an immobilized antibody that specifically binds to a T cell costimulatory molecule. Re-stimulation of the T-cells can occur within about eight to about twelve days of the initial stimulation of the T cells.

Stimulation of the CD4⁺ T cells is performed in the presence of a Th2 supportive environment, and the cells are allowed to proliferate in a Th2 supportive environment. In one embodiment, the Th2 supportive environment comprises at least 100 IU/ml of IL-4, for example at least 200 IU/ml IL-4, for example at least 500 IU/ml IL-4, for example at least 750 IU/ml IL-4, for example at least 1000 IU/ml IL-4. In another embodiment, the Th2 supportive environment further comprises no more than about 20 IU/ml of IL-2, for example no more than about 10 IU of IL-2, for example no more than about 5 IU of IL-2, for example no more than about 1 IU/ml of IL-2, or no IL-2. In another embodiment, the Th2 supportive environment further comprises between 1 and 20 IU/ml of IL-2, for example at least 1 IU/ml of IL-2 but no more than 20 IU/ml of IL-2, for example between 1 and 10 IU/ml of IL-2. In another example, the Th2 supportive environment further comprises rapamycin, such as at least 0.0004 μM, for example at least 0.004 μM, for example at least 0.02 μM, for example at least 0.1 μM.

In one embodiment, the substantially purified CD4⁺ Th2 lymphocytes secrete a Th2 cytokine. In another embodiment, the substantially purified CD4⁺ Th2 lymphocytes are substantially free of secretion of a Th1 cytokine. For example, the Th2 lymphocytes do not secrete measurable amounts of IL-2 but do secrete measurable amounts of IL-4. In a particular embodiment, the Th2 cells secrete IL-4, IL-5 and/or IL-10, but not a detectable amount of IL-2. In a particular embodiment, the purified CD4⁺ Th2 cells produce less than 10 pg/ml of IL-2 per 1×10⁶ CD4⁺ Th2 lymphocytes. In yet another embodiment, the Th2 lymphocytes produce at least 1000 pg/ml of IL-4 per 1×10⁶ CD4⁺ Th2 lymphocytes, such as at least 2000 pg/ml IL-4, for example at least 5000 pg/ml IL-4. The secretion of cytokines can be measured using standard bioassays, such as an ELISA. The purity of the population of CD4⁺ Th2 lymphocytes can be assessed by comparing the secretion profile with a control, such as a substantially purified population of purified CD4⁺ Th1 cells.

In one embodiment, the population of substantially purified cells produced has less than 5% Th1 lymphocytes, for example less than 1% Th1 lymphocytes. The proportion of Th1 lymphocytes in the population can be measured by any means known to one of skill in the art. For example, fluorescence activated cell sorting can be utilized. Alternatively the supernatant content is tested for secretion of cytokines. In one embodiment, an assay, such as a bioassay, and ELISA, or a radioimmuno assay, is performed to test the cytokine secretion profile of the cells.

The methods disclosed herein can further comprise cryo-preserving the purified CD4⁺ Th2 lymphocytes.

Also comprehended by this disclosure are CD4⁺ Th2 cells produced by the method disclosed herein.

### Methods for Treatment by Transplanting Purified/Expanded Th2 Cells

Donor T cells contained in a blood or marrow allograft mediate both beneficial and detrimental immune effects post-transplant. Although T cells mediate a potentially curative graft-versus-leukemia (GVL) effect and prevent marrow graft rejection, they can also generate graft-versus-host disease (GVHD). The relative balance of these immune effects is a primary determinant of clinical outcome after allogeneic transplantation. Clinical studies using T cell-depleted (TCD) marrow allografts clearly demonstrated the importance of T cell-mediated immune reactions after allogeneic bone marrow transplantation (alloBMT): recipients of TCD allografts had greatly reduced levels of GVHD, but had much higher rates of both graft rejection and leukemic relapse (Poynton, Bone. Marrow. Transplant. 3:265-79, 1988). Because TCD-alloBMT shifted the cause of mortality from GVHD to leukemia relapse and graft rejection, this approach did not represent a significant treatment advance relative to conventional T cell-replete alloBMT.

These observations have prompted investigation into the development of donor T cell administration methodologies which preserve an anti-leukemic effect and prevent graft rejection while limiting GVHD. Such methods include the administration of only CD4⁺ donor T cells (Champlin et al., Transplant. Proc. 23:1695-6, 1991), or the delayed administration of donor T cells post-transplant (Kolb et al., Blood. 76:2462-5, 1990). Both of these approaches have met with limited success, as leukemia relapse and significant levels of GVHD remain significant problems.

Disclosed herein is an alternative approach; donor T cells of defined cytokine phenotype are used to differentially mediate allogeneic transplantation responses. A type I immune response, mediated by CD4⁺, Th1 and CD8⁺, Tc1 cells, is characterized by the secretion of the pro-inflammatory cytokines IL-2 and IFN-γ (Mosmann et al., J. Immunol. 136:2348-57, 1986). In contrast, a type II immune response, mediated by CD4⁺, Th2 and CD8⁺, Tc2 cells, is characterized by the secretion of the anti-inflammatory cytokines IL-4, IL-5, and IL-10. Without being bound by theory, it was postulated that as acute GVHD is typical of a type I immune response (characterized by an initial phase of IL-2 production, and followed by IFN-γ secretion and cytolytic function) (Ferrara et al., N. Engl. J Med. 324:667-74, 1991) donor T cells of type II cytokine phenotype would regulate GVHD.

Thus, the administration of donor T cells of Th2 phenotype represents a novel strategy for the regulation of GVHD after allogeneic bone marrow transplantation. This treatment strategy is also of use in the treatment of solid tumors, such as carcinomas. The treatment strategy is further of use in the treatment of hematologic or lymphoid malignancies such as acute lymphocytic leukemia, acute myelogenous leukemia, chronic lymphocytic leukemia, chronic myelogenous leukemia, acute myelogenous leukemia, chronic lymphocytic leukemia, chronic myelogenous leukemia, indolent non-Hodgkin's lymphoma, high-grade non-Hodgkin's lymphoma, Hodgkin's lymphoma, multiple myeloma, or myelodysplastic syndrome. Specific, non-limiting examples of solid tumors that can be treated by the method disclosed herein include, but are not limited to, breast cancer, colon cancer, ovarian cancer, renal cell carcinoma, lung cancer, or melanoma.

Thus, a method of transplanting allogeneic donor immune cells to reconstitute immunity in a recipient with a hematologic or lymphoid malignancy or with a solid tumor is provided herein. The method includes depleting a recipient's T cells that mediate graft rejection. A therapeutically effective amount of a population of donor allogeneic cells comprising CD4⁺ and CD8⁺ T cells is administered to the recipient, as well as a therapeutically effective amount of a population of donor CD4⁺ Th2 cells. The method results in transplanting allogeneic immune cells into the recipient and reconstituting immunity in the recipient. In one example, the methods provided herein reduce morbidity and mortality which can from a transplant, such as GVHD.

The recipient's immune system, such as T cells, can be non-selectively or selectively depleted, or ablated, by any method known in the art, for example, selective depletion or ablation of T cells or a specific subset of T cells. In one embodiment, the recipient's T cells are depleted or ablated by the administration of an induction chemotherapy regimen which includes a therapeutically effective amount of etoposide, doxorubicin, vincristine, cyclophosphamnide, and prednisone (EPOCH). In another example, fludarabine can also be administered to improve the depletion of T cells. Allogeneic peripheral blood stem cell therapy (PBSCT) studies have demonstrated that fludarabine can contribute to the prevention of marrow rejection in non-myeloablative transplant approaches (Giralt et al., Blood 89:4531-6, 1997; Slavin et al., Blood 91:756-63, 1998). In the non-transplant setting, fludarabine administration can result in immunosuppression through its depletion of both CD4⁺ and CD8⁺ T cells (Cheson, J. Clin. Oncol. 13:2431-48, 1995); severe immune deficits are particularly observed when fludarabine is administered in combination with steroids (O'Brien et al., Blood 82:1695-1700, 1993), alkylating agents (Zaja et al., Eur. J. Haematol. 59:327-8, 1997), or topoisomerase II inhibitors (McConkey et al., J. Immunol. 156:2624-30, 1996). Patients receiving fludarabine-containing combination chemotherapy are susceptible to developing transfusion-associated GVHD (Williamson et al., Lancet 348:472-3, 1996). This observation illustrates the potential of fludarabine-based regimens for preventing the rejection of even HLA-disparate lymphoid cells. In the setting of T cell-replete HLA-matched PBSCT, fludarabine-based preparative regimens result in donor engraftment without myeloablation. However, allogeneic transplantation using such non-myeloablative regimens were still limited by a high incidence and severity of acute GVHD (Slavin et al., Blood 91:756-63, 1998; Khouri et al., J. Clin. Oncol. 16:2817-24, 1998; Giralt et al., Blood 89:4531-6, 1997).

Following depletion or ablation of the immune system, such as the recipient's T cells, a therapeutically effective amount of a population of donor allogeneic cells including CD4⁺ and CD8⁺ T cells are administered to the recipient. In one example, the donor is an HLA-matched donor. The donor allogeneic lymphocytes are collected by any method known to one of skill in the art. In one embodiment, the lymphocytes are collected by apheresis. In one specific non-limiting example, the lymphocyte fraction is collected by elutriation of the lymphocytes and depletion of the B cells. In another example, the lymphocyte fraction is collected by elutriation and enriched for CD34⁺ cells.

Substantially purified donor Th2 cells are prepared by the methods disclosed herein. A therapeutically effective amount of donor allogeneic cells including CD4⁺ and CD8⁺ T cells, and a therapeutically effective amount of a population of donor CD4⁺ Th2 cells, are administered to the recipient. Specific, non-limiting examples of a therapeutically effective amount of substantially purified CD4⁺ Th2 lymphocytes include substantially purified CD4⁺ Th2 lymphocytes administered at a dose of about 5×10⁶ cells per kilogram to about 125×10⁶ cells per kilogram, for example about 5×10⁶ cells per kilogram to about 25×10⁶ cells per kilogram, for example about 25×10⁶ cells per kilogram, for example about 125×10⁶ cells per kilogram.

The substantially purified donor CD4⁺ Th2 cells are administered at the same time, directly following, or at a time remote from the administration of the donor allogeneic cells including CD4⁺ and CD8⁺ T cells. In one specific non-limiting example, substantially purified Th2 cells are administered within one day of the donor allogeneic cells including CD4⁺ and CD8⁺T cells. In another specific, non-limiting example, the allogeneic cells including CD4⁺ cells and CD8⁺ cells are administered as peripheral blood stem cell therapy (PBSCT).

The substantially purified populations of CD4⁺ Th2 lymphocytes disclosed herein can be administered with a pharmaceutically acceptable carrier, such as saline. In one embodiment, compositions containing substantially purified populations of CD4⁺ Th2 lymphocytes can also contain one or more therapeutic agents, such as an anti-tumor agent, or non-cultured CD4+and CD8+T cells. Other therapeutic agents that can be used to practice the methods disclosed herein include, but are not limited to immune-depleting agents, such as a chemotherapeutic agent or a monoclonal antibody therapy. Such agents can be administered before, during or after administration of the Th2 cells, depending on the desired effect. In one embodiment, a population of substantially purified CD4⁺ Th2 lymphocytes from the subject is generated prior to administration of immune-depleting agents, and the Th2 cells administered subsequent to the administration of immune-depleting agents.

In one example, the dose of allogeneic CD4⁺ and CD8⁺ T cells administered to the subject is from about 40×10⁶T cells per kg to about 400×10⁶ T cells per kg. In another example, the dose of allogeneic CD4⁺ and CD8⁺ T cells is included in a peripheral blood stem cell transplant product In another embodiment, a method of treating a subject having an autoimmune disorder is provided. Specific, non-limiting examples of autoimmune disorders include rheumatoid arthritis, Crohn's disease, systemic lupus erythemetous, multiple sclerosis, and diabetes. The method for treating the autoimmune disorder includes utilizing immunosuppressive chemotherapy to deplete or ablate the T cells and B cells of the subject, as described above. A therapeutically effective amount of autologous peripheral blood cells including CD4⁺ and CD8⁺ T cells is administered to the subject, in addition to a therapeutically effective amount of autologous CD4⁺ Th2 cells. The administration of the autologous peripheral blood cells and autologous CD4⁺ Th2 cells results in the reconstitution of immunity in the subject, thereby treating the autoimmune disorder.

The method for treating the autoimmune disorder may involve the allogeneic transplant approach, as outlined above, or an autologous transplant approach. In the allogeneic method, the transfer of donor stem cells, donor CD4⁺ and CD8⁺ T cells, and donor Th2 cells may allow for fill donor engraftment with reduced GVHD. In this setting, replacement of the immune system of the subject with donor-type immunity alleviates one or more symptoms of the autoimmune disease.

In the autologous method to utilizing Th2 cells to treat autoimmunity, the subject's own immune system is reconstituted under the guidance of Th2 cells. This may reduce the severity of the autoimmune disease by changing the immune system of the patient from a Th1-type pro-inflammatory immunity to a Th2-type immunity with reduced inflammatory potential. In this method, the autoimmune disease patient is treated with immune-depleting chemotherapy such as the fludarabine and EPOCH regimen described herein. The therapy depletes or ablates the immune B and T cells that contribute to the autoimmune disease pathogenesis. After immune depletion, a therapeutically effective amount of autologous peripheral blood cells including CD4⁺ and CD8⁺ T cells is administered to the subject In addition, a therapeutically effective amount of autologous CD4⁺ Th2 cells are administered. The administration of the autologous peripheral blood immune cells and autologous CD4⁺ Th2 cells results in the reconstitution of immunity in the subject with an alteration of the immune cytokine phenotype towards a Th2-type profile, thereby treating the autoimmune disorder. In this autologous transplantation method of treating autoimmune disease, the dose of CD4⁺ and CD8⁺ T cells, and the dose of CD4⁺ Th2 cells is similar to that detailed above for allogeneic transplantation.

In addition to using Th2 cells to treat cancer and autoimmune diseases, Th2 cells can be used to facilitate solid organ transplantation. In this embodiment, the recipient has a disease of end-organ failure, such as lung failure, renal failure, pancreatic islet cell failure with resultant diabetes mellitus, heart failure, or liver failure. In this method, the treatment includes depletion or ablation of the T and B cells of the recipient with chemotherapy, such as the fludarabine and EPOCH regimen, followed by the administration of a therapeutically effective amount of allogeneic donor CD4⁺ and CD8⁺ T cells, and a therapeutically effective amount of donor CD4⁺ Th2 cells. Once donor immunity has been established in the recipient, the donor solid organ tissue can be administered to the recipient without the occurrence of solid organ allograft rejection. Thus, the donor solid organ tissue is HLA-matched to the allogeneic donor CD4⁺ and CD8⁺ T cells and the allogeneic donor CD4⁺ Th2 cells.

Disclosure of certain specific examples is not meant to exclude other embodiments. In addition, any treatments described in the specification are not necessarily exclusive of other treatment, but can be combined with other bioactive agents or treatment modalities.

### EXAMPLE 1

### Ex Vivo Generation of Donor CD4⁺ Th2 Cells

Lymphocyte Harvest and T Cell Isolation from Donor

A donor, such as an HLA-matched donor, underwent a 2 to 5 liter apheresis procedure using a CS-3000 or an equivalent machine to collect lymphocytes. The apheresis product was subjected to counterflow centrifugal elutriation by the standard operating procedure of the NIH DTM. The lymphocyte fraction of the elutriation product (120 to 140 fraction) was depleted of B cells by incubation with an anti-B cell antibody (anti-CD20; Nexell Therapeutics) and an anti-CD8 antibody (Nexell) and sheep anti-mouse magnetic beads (Dynal; obtained through Nexell) by a standard operating procedure of the NIH DTM using the MaxCep Device (Nexell). Cells isolated by this type of procedure have been infused without any toxicity that can be attributed to the selection procedure. Flow cytometry was performed to document that CD8⁺ T cell contamination was <1%. The resultant CD4-enriched donor lymphocyte product was cryopreserved using an NIH DTM protocol in aliquots of 50 to 200×10⁶ cells/vial. Sterility of the population was not tested at this early stage of the Th2 cell generation procedure; such testing occurred after the final co-culture of donor CD4 cells with recipient antigen presenting cells (APC).

ACK lysis buffer (BioWhitaker) was used to initially remove red blood cells from the cell product. Hanks Balanced Salt Solution was used in the cell processing as a wash buffer. All of these media are ancillary reagents in the Th2 generation process, as they are washed out prior to final Th2 cell cryopreservation.

Ex vivo Generation of CD4⁺ Th2 Cells

Cryopreserved donor CD4⁺ T cells were resuspended to a concentration of 0.3×10⁶ cells per ml. Media contained of X-Vivo 20 (BioWhitaker) supplemented with 5% heat-inactivated autologous plasma (herein referred to as “media”). The donor CD4⁺ T cells were cultured in filtered flasks at 37° C. in 5% CO₂ humidified incubators. At the time of culture initiation, T cells were stimulated with anti-CD3/anti-CD28 coated magnetic beads (3 to 1 ratio of beads to T cells). Tosylated magnetic beads (Dynal) are conjugated with an antibody to human CD3 (clone OKT3) and an antibody to human CD28 (clone 9.3). In 50 infusions of T cells grown with anti-CD3/anti-CD28 coated beads, there have been no adverse except the development of an asymptomatic HAMA serologic response in one patient.

At the time of co-culture initiation and on day two of culture, the following reagents were added: recombinant human IL-4 (obtained through cross-filing on CTEP IND of Shering IL4; 1000 I.U. per ml; target specific activity is 2.67×10⁷ I.U. per mg.), and recombinant human IL,2 (Chiron Therapeutics; 20 I.U. per ml), or IL-4 without IL-2. IL-4 may be obtained by completing a Clinical Drug Request (NIH Form #986) and mailing it to the Drug Management and Authorization Section, PMB, DCTD, NCI, 9000 Rockville Pike, EPN 707, Bethesda, Md., 20892-7422.

After day 2, cells were maintained at a concentration of 0.25 to 1.0×10⁶ cells per ml by the addition of fresh X-Vivo 20 media supplemented with autologous plasma (5%), IL-2 (20 I.U./ml), and IL-4 (1000 I.U./ml). The median cell volume was determined using a Multisizer II instrument (Coulter). When the T cell volume approached 500 fl (acceptable range of 650 to 350), the T cells were restimulated with anti-CD3/anti-CD28 beads. Typically, this time of restimulation was after 8 to 12 days of culture. Bead restimulation was at a bead to T cell ratio of 3:1. T cell concentration was 0.2×10⁶ cells/ml. Media again contained X-Vivo 20 supplemented with autologous plasma (5%), IL-2 (20 I.U./ml), and IL-4 (1000 I.U./ml), or media with IL4 but without IL-2.

After bead re-stimulation, CD4 cells were maintained at a concentration of 0.25 to 1.0×10⁶ cells per ml by the addition of fresh X-Vivo 20 media supplemented with autologous plasma (5%), IL-2 (0 or 20 I.U./ml), and IL-4 (1000 I.U./ml). When the CD4 cell mean cell volume approached 500 fl (acceptable range of 650 to 350), the cells were harvested and cryopreserved by the NIH DTM method in protocol-relevant quantities for administration. Generally the total time of CD4 cell culture was 15 to 20 days.

### EXAMPLE 2

### Demonstration of Th2 Cell Expansion

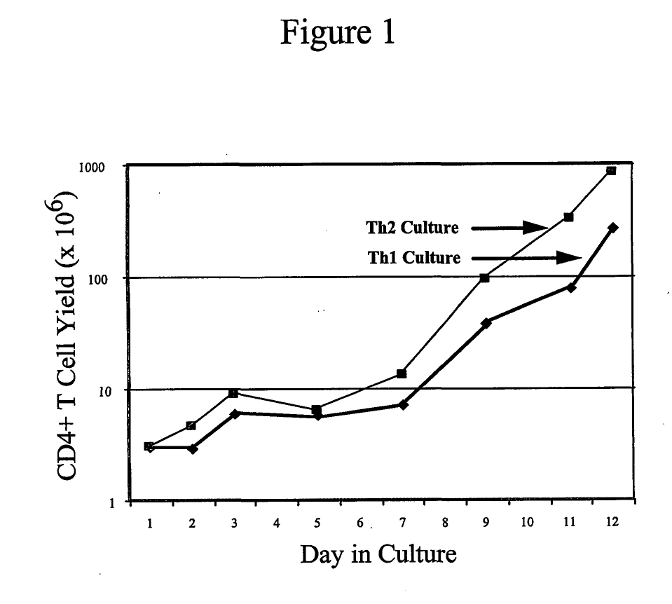

Human CD4⁺ cells from a stem cell transplant donor were stimulated ex vivo as described in Example 1. Briefly, human peripheral blood lymphocytes were collected by apheresis and subsequently purified by counterflow centrifugal elutriation. CD4⁺ T cells were enriched for by negative selection using anti-CD8 and anti-CD20 antibodies and sheep anti-mouse magnetic beads. Two rounds of antibody depletion were performed to ensure that CD8⁺ T cell content was less than 0.5% of the staring T cell population. CD4-enriched T cells were plated in tissue culture flasks at a concentration of 200,000 cells per ml of culture media, which included of X-Vivo 20 media supplemented with 5% autologous plasma. Anti-CD3, anti-CD28 coated magnetic beads were added to the culture at a T cell to bead ratio of 1:3. In the Th2 culture flask, recombinant human IL-2 (20 I.U./ml) and recombinant human IL-4 (1000 I.U./ml) were added; in the Th1 culture flask, recombinant human IL-2 (1000 I.U./ml), recombinant human IL-12 (2.5 ng/ml), and a neutralizing amount of an antibody to IL-4 were added. The growth of the cells was evaluated over time. As shown in FIG. 1, CD3/CD28 stimulation resulted in CD4⁺ cell expansion in both the Th2 and Th1 culture conditions.

### EXAMPLE 3

### Cytokine Secretion Profile of Th2 Cells

Cells were prepared as described in Example 2. Th1 and Th2 cultures were maintained at a concentration of 200,000 cells per ml by the addition of fresh media replete with recombinant cytokines. Cultures were monitored for cell volume by Coulter multisizer analysis. When the cell volume approached 650 fl (typically 8 to 12 days in culture), the Th1 and Th2 cells were harvested and restimulated with anti-CD3, anti-CD28 coated beads (1:3 ratio), and further expanded in cytokine-containing media. When the cell volume again returned to approximately 650 fl (typically after an additional 7 days in culture), the cells were restimulated with CD3, CD28-coated beads and a 24 hour supernatant was generated. The Th1 or Th2 supernatant was subsequently analyzed for cytokine content by two-site ELISA technique (BioSource).

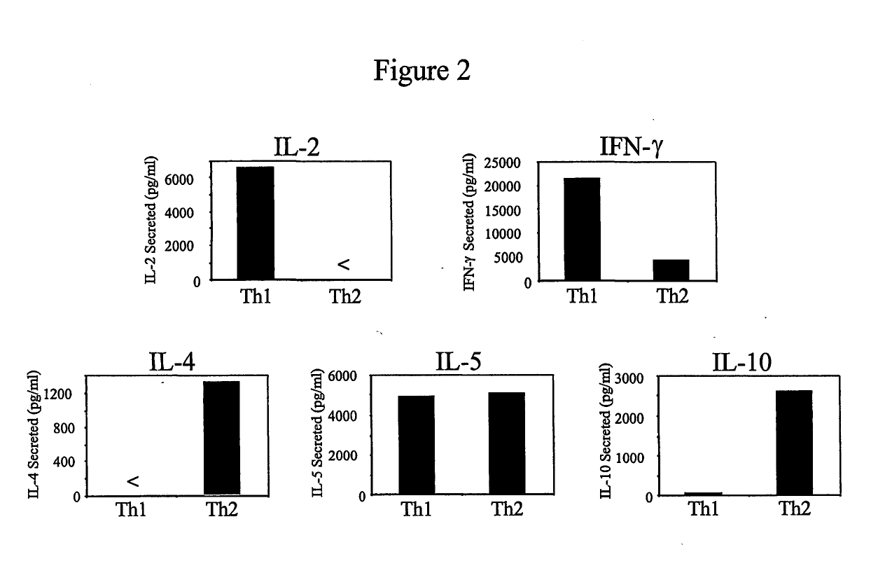

As FIG. 2 demonstrates, CD4 cells propagated in the Th1 culture condition produced a high level of the type I cytokines IL-2 and IFN-γ upon repeat CD3, CD28 stimulation In contrast, the CD4 cells propagated in the Th2 culture condition produced an undetectable level of IL-2 and a reduced level of IFN-γ. This indicates that the Th1 culture produced a greater level of type I cytokines than the Th2 culture. In comparison, the Th2 culture secreted a high level of the type II cytokine IL-4, whereas the Th1 culture did not secrete a detectable level of IL-4. Similarly, the Th2 culture produced an increased amount of the type II cytokine IL-10 relative to the Th1 cells.

Therefore, using the Th1 and Th2 culture conditions described herein, CD3/CD28 stimulation of purified human CD4⁺ T cells can be utilized to generate Th1 or Th2 cells. Th1 cells are characterized by their secretion of type I cytokines, such as IL-2 and IFN-γ and their reduced level of secretion of type II cytokines, such as IL-4 and IL-10. Th2 cells are characterized by their secretion of the type II cytokines and their reduced level of secretion of the type I cytokines.

### EXAMPLE 4

### Purification of the CD4⁺RO⁺ subset of CD4⁺ cells Enhances Th2 Cell Generation

Purified CD4⁺ T cells obtained using the methods disclosed above were further purified into the CD4⁺RA⁺ T cell subset (naïve subset) or the CD4⁺RO⁺ T cell subset (memory-type subset). This extra purification step was performed using a positive selection method in which monoclonal antibodies specific for the RA and RO antigens on CD4 cells (PharMingen, Inc.; CD45RA antibody catalog #555488 and CD45RO antibody catalog #555492) were used. After marking the RA and RO subsets of CD4 cells, each population was subsequently purified by flow sorting using a FACSort machine (Becton Dickinson Immunocytometry Systems).

Purified CD4^{'} RA⁺ and CD4⁺RO⁺ subsets of CD4 cells were subjected to the Th1 and Th2 culture conditions as detailed in the above examples. Briefly, the RA and RO cells were cultured separately in the Th1 stimulating environment (CD3, CD28 stimulation in the presence of 1000 I.U./ml of IL-2, 2.5 ng/ml of IL-12, and the anti-IL-4 monoclonal antibody), or the Th2 stimulating environment (CD3, CD28 stimulation in the presence of 1000 I.U./ml of HA and 20 I.U./ml of IL-2). After 10 days in culture, each of the four cultures were harvested and re-stimulated with CD3, CD28 beads (1:3 ratio of T cells to beads). A 24 hour supernatant was generated, and tested for cytokine content by two-site ELISA.

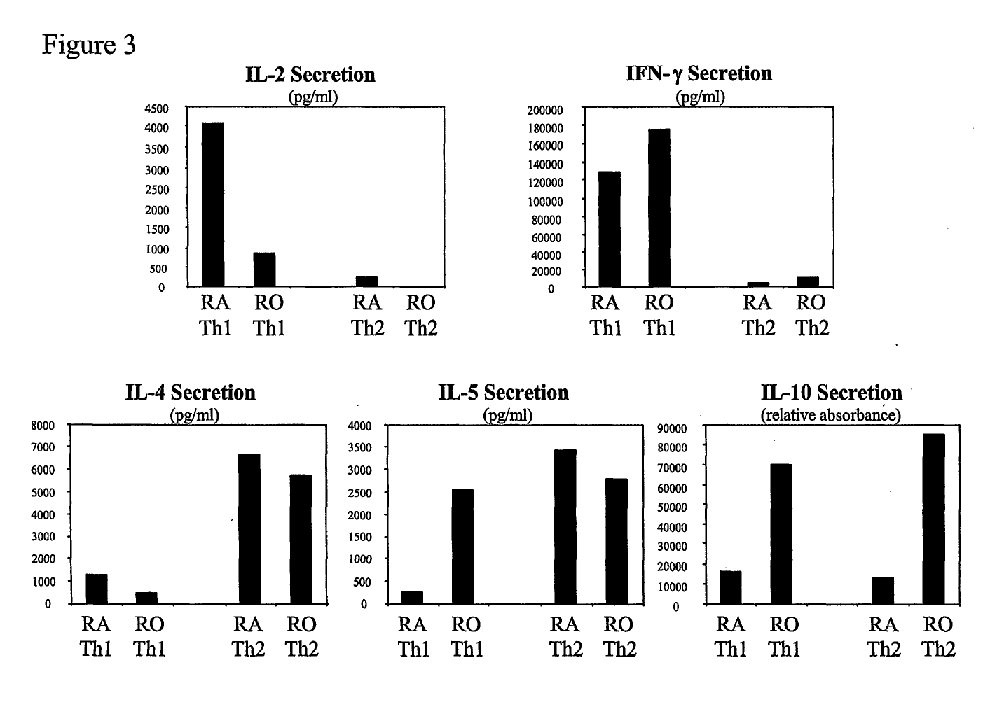

As shown in FIG. 3, the CD4⁺RO⁺ subset cultured in the Th2 supportive environment had higher Th2 purity relative to the CD4+RA subset. That is, relative to the Th2 culture condition using CD4⁺RA⁺ cells, the CD4⁺RO⁺ Th2 culture increased secretion of the type II cytokine IL-10 and a comparable level of the type II cytokines IL-4 and IL-5. Furthermore, relative to the Th2 culture condition using CD4⁺RA⁺ cells, the CD4⁺RO⁺ Th2 culture demonstrates a reduced secretion of type II cytokine IL-2, and a comparable level secretion of the type I cytokine INF-γ. Therefore, the RO subset generated a purer Th2 phenotype (increased Th2-type cytokine secretion and decreased Th1-type cytokine secretion). In addition, the Th2 cells generated from the CD4⁺RO⁺ starting cell population had a greatly enriched Th2 cytokine profile relative to the control Th1 cultures initiated from the RA⁺ or RO⁺ cell subsets.

Similarly, the CD4⁺RA⁺ subset cultured in the Th1supportive environment had higher Th1 purity relative to the CD4+RO subset. That is, relative to the Th1 culture condition using CD4⁺RO⁺ cells, the CD4⁺RA⁺Th1 culture increased secretion of the type I cytokine IL-2 and a comparable level of the type I cytokine IFN-γ. Furthermore, relative to the Th1 culture condition using CD4⁺RO⁺ cells, the CD4⁺RA⁺ Th1 culture demonstrates a reduced secretion of type II cytokines IL-5 and IL-10, and a comparable level secretion of the type II cytokine IL-4. Therefore, the RA subset generated a purer Th1 phenotype (increased Th1-type cytokine secretion and decreased Th2-type cytokine secretion). In addition, the Th1 cells generated from the CD4⁺RA⁺ starting cell population had a greatly enriched Th1 cytokine profile relative to the control Th2 cultures initiated from the RA⁺ or RO⁺ cell subsets.

These results demonstrate that generation of the Th2 subset can be enhanced by further purification of-the CD4⁺RO⁺ subset of CD4 cells and that the generation of the Th1 subset can be enhanced by further purification of the CD4⁺RA⁺ subset of CD4 cells.

### EXAMPLE 5

### Effect of Immuno-Suppression Drugs on Th2 Cell Generation

To determine the effect of immune suppression agents on Th2 cell generation, murine splenic CD4⁺ T cells were purified and stimulated in a Th2 stimulating environment, in the presence or absence of rapamycin or cyclosporine A (CSA). One skilled in the art will understand that similar methods can be used to test other immuno-suppressive agents. In addition, using the disclosure provided in the above examples, similar experiments can be performed on human CD4⁺ T cells.

Murine splenic CD4⁺ T cells were purified by negative selection from C57B1/6 mice, and co-stimulated with anti-CD3, anti-CD28 coated magnetic beads as described in the above examples. Cytokine culture conditions were optimized for murine Th2 cell generation. The Th2 cell culture conditions included RPMI-1640 with 10% fetal calf serum, 1000 I.U./ml of recombinant murine IL-4 (Peprotech, Rocky Hill, N.J.), 20 I.U./ml recombinant human IL-2, 20 ng/ml recombinant human IL-7, and 3.3 μM of N-acetyl cysteine. In some experiments, the cells were further incubated with rapamycin (rapa, 0.1 or 0.02 μM, Sigma, St. Louis, Mo.) or CSA (0.2 or 0.04 μM, Sigma). Rapamycin and CSA were present from the initiation of the culture, and the cells received only the anti-3/anti-28 stimulation on day 0 (no re-stimulation).

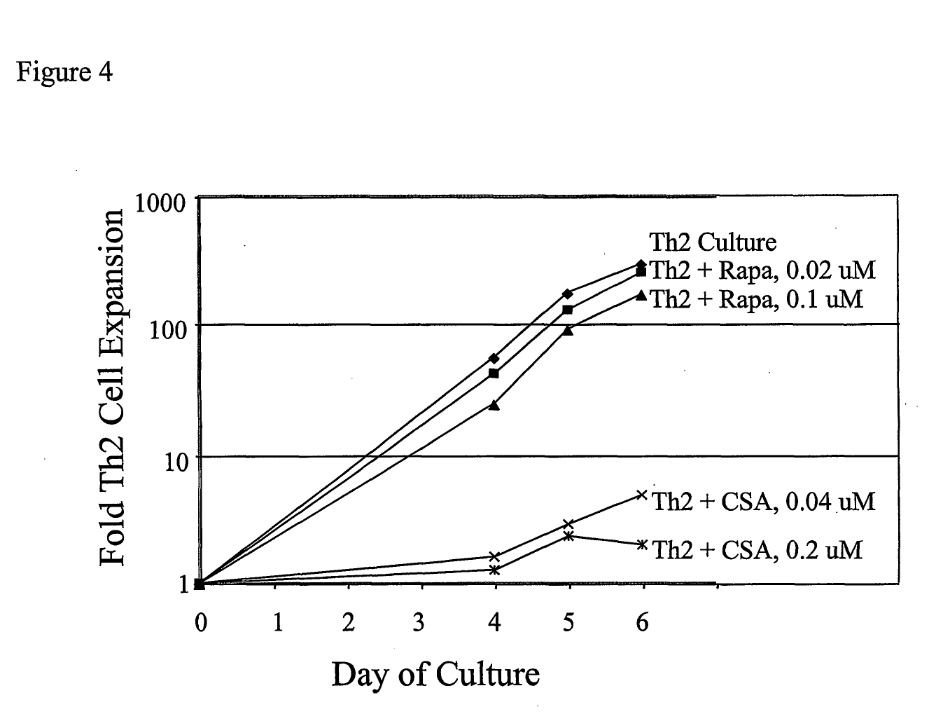

As shown in FIG. 4, Th2 expansion in rapa is preserved. In contrast, CSA suppresses Th2 cell expansion. These results indicate that rapa may be preferable for use as an immune suppression agent relative to CSA, because it promotes Th2 instead of suppressing it.

The effect of CSA and rapa on cytokine secretion in the cells was also examined using the ELISA cytokine secretion assay described above in Examples 3 and 4. Briefly, murine CD4⁺ T cells were co-stimulated with anti-CD3, anti-CD28 in the Th2 culture conditions described above, in the absence or presence of rapa (0.1 μM) or CSA (0.2 μM). After 6 days of culture, the CD4 cells were restimulated with anti-CD3, anti-CD28 in fresh media not containing cytokines or immune suppression molecules, and the 24 hour supernatant was tested for the Th1 or Th2-type cytokines by ELISA.

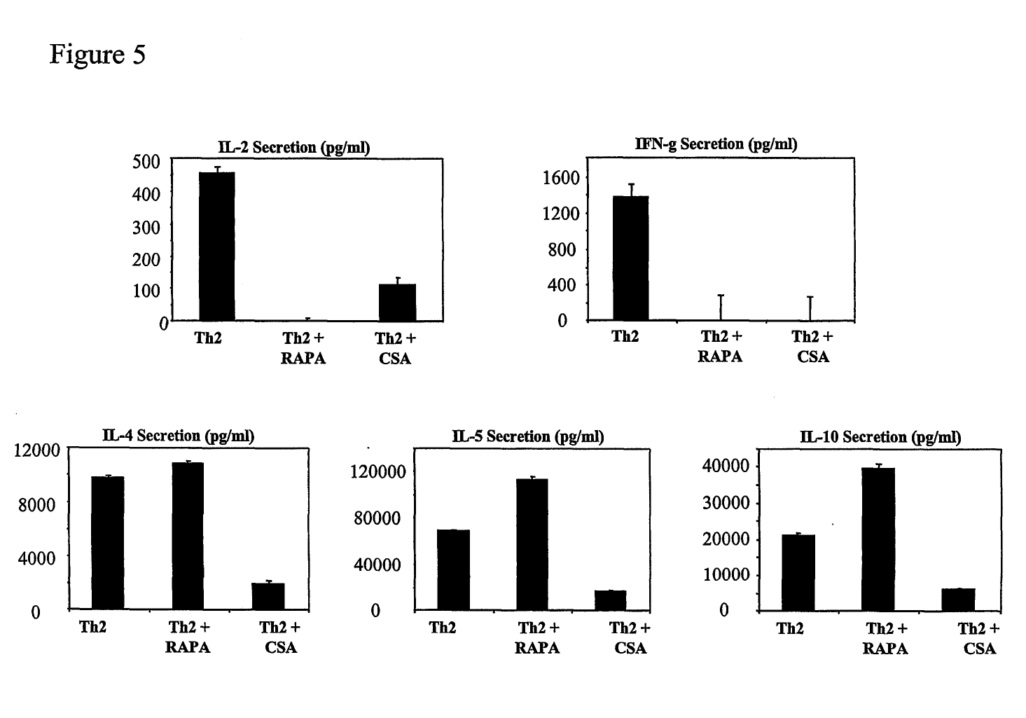

As shown in FIG. 5, Th2 cells grown with 3/28 in the presence of rapamycin have a greatly enhanced Th2 profile. The rapa-Th2 cells secrete very low levels of IL-2 and IFN-γ, and stable or enhanced type-II cytokines IL-4, IL-5, and IL-10. In marked contrast, Th2 cells grown in CSA have a reduced of both type-I and type-II cytokines. These results indicate that rapamycin can synergize with the 3/28 methods described herein to further promote Th2 cell generation, and that rapamycin may be a more appropriate agent to administer after an allogeneic transplantation that involves Th2 cells, since it does not reduce Th2-type cytokines.

### EXAMPLE 6

### Clinical Trial to Evaluate Use of Donor Th2 Cells for the Prevention of GVHD in Non-Myeloablative, HLA-Matched Allogeneic Peripheral Blood Stem Cell Transplantation: Donor and Recipient Qualifications

Inclusion Criteria: Patient

Patients with lymphoid malignancy and leukemia (including myelodysplasia) were candidates for this study. The following diagnoses and ages were considered (Table 1):

TABLE 1
Patient Inclusion Criteria

| Disease | Disease Status | | Age |
|---|---|---|---|
| Chronic Lymphocytic | a) | Relapse Post- | 18 to 75 |
| Leukemia | | fludarabine, or | |
| | b) | Non-complete | |
| | | remission (CR) after | |
| | | Salvage Regimen. | |
| Hodgkin's and | a) | Primary Treatment | 18 to 75 |
| Non-Hodgkin's Lymphoma | | Failure | |
| (all types, including | b) | Relapse after | |
| Mantle Cell Lymphoma) | | AutoSCT, or | |
| | c) | Non-CR after Salvage | |
| | | Regimen | |
| Multiple Myeloma | a) | Primary Treatment | 18 to 75 |
| | | Failure, or | |
| | b) | Relapse after | |
| | | AutoSCT, or | |
| | c) | Non-CR after Salvage | |
| | | Regimen | |
| Acute Myelogenous | a) | In CR #1, 2 or 3 | 18 to 75 |
| Leukemia | b) | Any Relapse with less | |
| | | than 10% blasts in | |
| | | marrow and blood. | |
| Acute Myelogenous | a) | In Complete Remission | 55 to 75 |
| Leukemia | | #1 or 2; or | |
| | b) | Any Relapse with less | |
| | | than 10% blasts | |
| | | in marrow and blood. | |
| Acute Lymphocytic | a) | In Complete Remission | 18 to 75 |
| Leukemia | | #2 or #3; or | |
| | b) | Any Relapse with less | |
| | | than 10% blasts | |
| | | in marrow and blood. | |
| Myelodysplastic | a) | RAEB | 18 to 75 |
| Syndrome | b) | RAEB-T (if blasts | |
| | | are <10% in marrow and | |
| | | blood after induction | |
| | | chemotherapy) | |
| Chronic Myelogenous | a) | Chronic Phase CML | 50 to 75 |
| Leukemia | b) | Accelerated Phase CML | |

Ideally, patients were at least 16 and not greater than 75 years old. Recipients ideally had a 5/6 or 6/6 antigen (A, B, and DR loci) HLA-matched first degree relative donor; Karnofsky performance

status of >70% (see Table 2); life expectancy >3 months; serum bilirubin <2.5 mg/dL, and serum ALT and AST values less than or equal to 2.5 times the upper limit of normal. Values above these levels can be accepted, if such elevations were thought to be due to tumor involvement by the lymphoid malignancy. If these values do not normalize during the induction chemotherapy, such patients were not eligible for the transplant phase of the protocol, and were taken off the study. Recipients also ideally had a creatinine clearance ≧60 mi/min or serum creatinine of ≦1.5 mg/dl; DLCO >50% of predicted; left ventricular ejection fraction of ≧45% by MUGA or ECHO; ability to give informed consent and durable power of attorney form completed.

TABLE 2
Karnofsky Scores

| | Karnofsky Score |
|---|---|
| Asymptomatic and fully active | 100% |
| Symptomatic; fully ambulatory; restricted | 80-90% |
| in physically strenuous activity | |
| Symptomatic; ambulatory; capable of | 60-70% |
| self-care; >50% of waking hours are | |
| spent out of bed | |
| Symptomatic; limited self-care; | 40-50% |
| spends >50% of time in bed, but not | |
| bedridden | |
| Completely disabled; no self-care; | 20-30% |
| bedridden | |

Exclusion Criteria: Patient

Subjects were excluded from the clinical trail if they had an infection that was not responding to anti-microbial therapy, had active CNS involvement by tumor, were HIV positive, hepatitis B or C surface antigen positive, were lactating or pregnant females (due to risk to fetus or newborn), or had a history of a psychiatric disorder which could compromise compliance with transplant protocol, or which did not allow for appropriate informed consent

Inclusion Criteria: Donor

Donors were a first-degree relative matched with recipient at 5/6 or 6/6 of the major HLA loci (A, B, and DR loci). Ideally, donors had adequate venous access for peripheral apheresis, or consent to use a temporary central venous catheter for apheresis. Donors should be at least 12 years of age and have the ability to give informed consent. Ideally, donors had no history of uncontrolled hypertension, stroke, or severe heart disease; had Hb of 11 gm/dl or greater, and platelet count of 100,000 per μl or greater.

Exclusion Criteria: Donor

Donors were excluded from the clinical trial if they had a history of a psychiatric disorder which could compromise compliance with transplant protocol, or which did not allow for appropriate informed consent; had a history of hypertension that was not controlled by medication, stroke, or severe heart disease; symptomatic angina, or a history of coronary artery bypass grafting or angioplasty or considered to have severe heart disease; anemia (Hb less than 11 gm/dl) or thrombocytopenia (PLT less than 100,000 per μl); lactating or pregnant females; or were HIV, Hepatitis B or C antigen positive.

Research Evaluation

All patients and donors were screened by complete history and physical examination. The following laboratory serologic evaluations were performed on the transplant recipient: typing for HLA-A, -B, and -DR (donor and patient); unilateral bone marrow aspirate and biopsy; cytogenetics and flow cytometry performed on marrow aspirates if disease could be followed by that modality; PCR test of DNA mini-satellite regions for future determination of chimerism; antibody screen for hepatitis A, B, and C; HIV, HTLV-I/II, CMV, adenovirus, EBV, HSV, toxoplasma, and syphilis (donor and patient); PPD test (optional, performed in individuals considered to be in a high-risk group); CBC with differential, PT, and PTT, and ABO typing (donor and patient); urine BHCG in females (donor and patient); and acute care, hepatic, and mineral panel.

The following radiologic, nuclear medicine, and special studies were also performed on the transplant recipient: chest radiographs; pulmonary function tests (vital capacity, FEV-1, DLCO); CT scans of chest, abdomen, and pelvis; CT or MRI of the head for all patients; cardiac tests: EKG, MUGA scan; skeletal survey (for Multiple Myeloma patients only). All radiological studies that identify measurable disease were repeated after each cycle of induction chemotherapy.

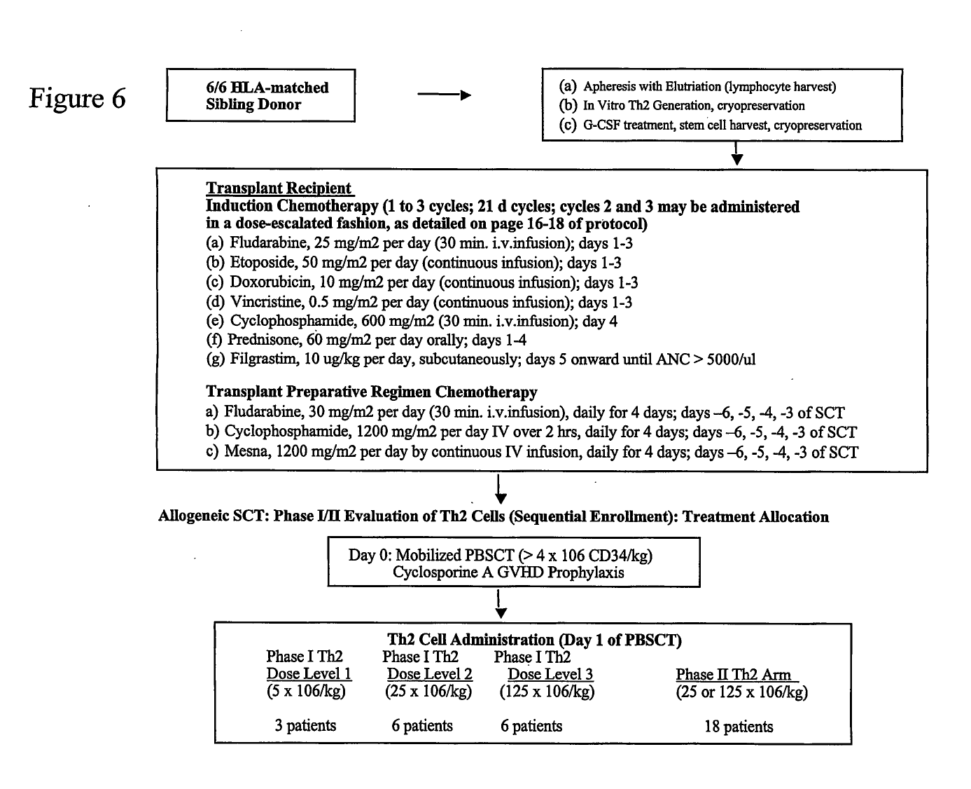

There was no randomization for the pilot study. Patients were sequentially enrolled to one of three Th2 cell dose levels, as shown in FIG. 6. After completion of the phase I Th2 component (Th2 cells administered at 5, 25, or 125×10⁶ cells/kg), 18 additional patients received Th2 cells at either dose level #2 (25×10⁶ cells/kg) or dose level #3 (125×10⁶ cells/kg) as part of the phase II Th2 component. The initial clinical results are shown below.

The overall study design is shown in FIG. 6.

### EXAMPLE 7

### Generation and Administration of Th2 Cells and Harvest and Administration of PBSC

The Th2 cells of the present disclosure can be used to generate a type II cytokine profile in a subject, thereby reducing or eliminating GVHD after allogeneic bone marrow transplantation, treating tumors and/or treating an autoimmune disorder. Administration of Th2 cells has been shown to reduce GVHD in subjects receiving an allogenic bone marrow transplant, while preserving the beneficial ability of donor T cells to prevent allograft rejection. Therefore, administration of Th2 cells to a subject in these clinical settings can improve the subject's response to a transplant.

Using the methods disclosed above, Th2 cells obtained from a subject were purified and expanded ex vivo. The expanded Th2 cells were introduced at a therapeutically effective dose into the same or another subject to stimulate a subject's immune system toward a type II cytokine profile.

Lymphocyte Harvest and T Cell Isolation from Subject

Blood was collected from a subject, such as an HLA-matched donor, and a substantially purified population of Th2 cells generated, using the method disclosed in EXAMPLE 1. The subject need not receive any particular treatment prior to harvesting the CD4⁺ cells. Briefly, the subject underwent a 2 to 5 liter apheresis procedure using a CS-3000 or an equivalent machine. The apheresis product was subjected to counterflow centrifugal elutriation, and the lymphocyte fraction (120 to 140 fraction) depleted of B cells by incubation with an anti-B cell antibody, an anti-CD8 antibody and sheep anti-mouse magnetic beads. Flow cytometry was used to demonstrate that CD8⁺ T cell contamination was <1%.

The resultant CD4⁺-enriched lymphocyte product can be cryopreserved using standard methods (for example using a combination of Pentastarch and DMSO) in aliquots of 50 to 200×10⁶ cells/vial. To qualify for cryopreservation, the cell culture should contain predominately CD4⁺ T cells by flow cytometry (greater than 70% CD4^{'} T cells, and less than 5% contaminating CD8^{'} T cells). Sterility of the population was not be tested at this stage of the Th2 cell generation procedure; such testing occurred after the final co-culture of donor CD4 cells with recipient APC.

Peripheral Blood Stem Cell (PBSC) Harvest

Immediately following lymphocyte harvest, the donor subject received filgrastim as an outpatient (10 ug/kg/day each morning; subcutaneously) for 5, 6, or 7 days. The subject should take the filgrastim as early as possible upon awakening in the morning. This is especially important on days 5, 6, and 7 of the injections.

Apheresis was typically performed on days 5 and 6. On some occasions, sufficient numbers of CD34⁺ cells were obtained with a single apheresis on day 5; on other occasions, apheresis was performed on days 5, 6, and 7 to reach the target CD34⁺ cell number (≧4×10⁶ per kg). The subject was instructed to take filgrastim for the complete 7 day period, unless notified by the transplant team that adequate CD34⁺ cells were harvested before day 7.

If ≧3×10⁶ CD34⁺ cells per kg were harvested after apheresis on days 5, 6, and 7, no further mobilization or apheresis was performed, and the recipient is eligible to receive the stem cell transplant with that dose of CD34⁺ cells.

When less than 3×10⁶ CD34⁺ cells per kg are harvested after apheresis on days 5, 6, and 7, the subject was given two weeks of rest, and then re-treated with filgrastim followed by repeat peripheral blood stem cell harvesting.

A 15 to 25 liter large volume whole blood apheresis was performed via a 2-armed approach or via a temporary central venous catheter in the femoral position using the Baxter CS3000Plus, Cobe Spectra, or an equivalent instrument. This procedure typically took 4 to 6 hours.

Apheresis procedure uses ACD-A anti-coagulant; alternatively, partial anti-coagulation with heparin is utilized. The apheresis product was cryopreserved and stored at −180° C. in a solution containing Plasmalyte A, Pentastarch, human serum albumin, DMSO, and preservative free heparin (10 U/ml). The concentration of CD34⁺ cells in the apheresis product was determined by flow cytometry, and the number of CD34⁺ cells in each cryopreserved bag calculated. If the donor and host are ABO incompatible, red blood cells are depleted from the stem cell product by standard protocols.

Ex vivo Generation of CD4⁺ Th2 Cells

The cryopreserved CD4⁺ T cells were resuspended to a concentration of 0.3×10⁶ cells per ml, and expanded using the method disclosed in Examples 1-3. The resulting population of substantially purified Th2 cells can be used immediately, or cryopreserved for future use. To qualify for cryopreservation with subsequent administration, the Th2 cell culture ideally contained predominantly (>70%) CD4⁺ T cells and less than 5% CD8^{'} T cells. For example, the population of substantially purified Th2-cells is at least 70%, 75%, 80%, 85%, 90%, 95%, 98%, or even at least 99% pure.

In addition, the T cells were tested for fungal and bacterial cultures, using standard testing done on cell products and for endotoxin content, using a limulus assay. Cell products positive for fingal, bacterial, or endotoxin content were discarded. T-cells obtained from subjects infected with HIV will also be infected with HV, as the virus directly infects CD4⁺ T cells. Therefore, in samples obtained from HIV positive subjects, methods can be used to control HIV infection during CD4 propagation, such as administration of anti-HIV drugs to the culture or gene-transfer approaches.

To estimate the number of Th2 cells that can be obtained from a subject, the following calculations can be used as a guideline. About 0.5×10⁶ CD4⁺ T cells can be harvested from one ml of blood. Assuming a 2-log expansion of Th2 cells in culture, it is estimated that 4×10⁷ Th2 cells could be generated from one ml of blood. This value assumes 100% efficiency at each step of the process, which is likely not to occur; a range of 20-100% efficiency is reasonable. Therefore, about 0.8-4×10⁷ Th2 cells could be generated per ml of blood.

Administration of Generated Th2 Cells

On day 1 of the transplant procedure, Th2 cells were administered intravenously. If the Th2 cells were previously cryopreserved, the cells were thawed and diluted in saline solution to a volume of approximately 125 to 250 ml for intravenous infusion. The dose of Th2 cells for each of the three Th2 cell dose levels is shown in FIG. 6. Steroids were not administered to manage DMSO-related toxicities (chills, muscle aches) that may occur immediately after cellular infusion (diphenhydramine and meperidine are allowed). The determination of whether a Th2 cell infusion was safe is based on the presence or absence of hyperacute GVHD and of any grade 4 or 5 toxicity attributable to the Th2 cells that occurs in the first 14 days post-transplant Hyperacute GVHD is defined as a severe level of acute GVHD (grade 3 or 4) that occurs within the first 14 days post-transplant.

Th2 cells can be administered in one or more pharmaceutically acceptable carriers, such as a saline solution. In addition, the Th2 cells can be administered concurrently (or separately) with other therapeutic agents, such as anti-microbial agents, and/or anti-tumor agents. In addition to administering substantially purified Th2 cells, non-cultured CD4⁺ and CD8^{'} T cells can be administered with the Th2 cells (concurrently or separately), allowing a more complete CD4^{'} and CD8^{'} immune recovery in a CD4^{'} Th2 and a CD8⁺ Tc2 manner. For example, patients received the stem cell transplant (which is T cell replete and therefore contains non-cultured CD4 and CD8 cells) on day 0 of the transplant On day 1 of the transplant, the patient received the ex vivo generated Th2 cells.

Examples of subjects who would benefit from such therapy include, but are not limited to, those receiving a stem cell or solid organ transplant those having an autoimmune disorder, and those having at least one tumor.

In a particular example, the dose of Th2 cells administered to a subject was in the range of: dose #1, about 5×10⁶ Th2 cells/kg; dose #2, about 2.5×10⁷ Th2 cells/kg; dose #3, about 1.25×10⁸ Th2 cells/kg. Ideally, no cortico-steroids were administered in the management of DMSO-related toxicities (chills, muscle aches) that may occur immediately after cellular infusion (diphenhydramine and meperidine are instead administered). The subject was monitored for the presence or absence of any grade 4 or 5 toxicity attributable to the Th2 cells that can occur in the first 14 days post-transplant Toxicity was monitored by criteria established by the National Cancer Institute Cancer Therapy and Evaluation Program (NCI-CTEP). Grade 4 toxicity is considered “life-threatening” whereas Grade 5 toxicity is death. Each organ system (GI system, renal system, nervous system, etc.) is graded on the grade 0 (not observed) to grade 5 scale (see also Table 7).

If no grade 4 or 5 toxicity attributable to the Th2 cells is observed in an initial three subjects receiving a particular dose of Th2 cells, then it is determined that that dose level has acceptable toxicity, and accrual to a higher dose level commences. For example, if no grade 4 or 5 toxicity attributable to the Th2 cells is observed in an initial three subjects receiving dose #1, then it is determined that dose level #1 has acceptable toxicity, and accrual to dose level #2 commences. If grade 4 or 5 toxicity attributable to the Th2 cells is observed in any of the initial three subjects, then accrual to dose level #1 is expanded to include a total of six patients. If two subjects in six develop a grade IV toxicity related to the Th2 cells, then it is determined that dose level #1 is not acceptable, and further accrual to the study stops at that point. If only one of the six patients experiences such an adverse effect, then it is determined that dose level #1 has acceptable toxicity, and accrual proceeds to dose level #2.

Three subjects are then subjected to Th2 cell dose level #2 (2.5×10⁷ Th2 cells/kg). The same accrual and stopping rules apply to this dose level as those used for dose level #1. As such, either three or six subjects are accrued to dose level #2.

If it is determined that Th2 cell dose level #2 has acceptable toxicity, accrual to the final dose level #3 starts (Th2 cell dose of 1.25×10⁸ cells/kg). Six subjects are evaluated on dose level #3. If m than one subject on dose level #3 develops a grade 4 or 5 toxicity attributable to the Th2 cells, then accrual to dose level #3 stops. Attempts were made to space patient accrual to help ensure that the safety and GVHD results from Th2 dose level #3 were available prior to the need to transplant the first patient in the phase II component of the study.

The Th2 cells disclosed herein can be administered to a subject one or more times as necessary for a particular subject Although one infusion may be sufficient, several infusions can be performed to increase the benefit, as some tumors and GVHD are oftentimes chronic and difficult to treat If multiple infusions are performed, they can be separated by a period of about four weeks. During such treatment, the patient is monitored, for example by performing tests about once or twice during each 4 week treatment cycle. Tests would include measurement of T cell cytokines, measurement of immune recovery panels such as T cell counts and T cell diversity and competence using methods known to those skilled in the art. In addition, tests that measure disease activity can also be performed to monitor the beneficial effect of the Th2 cells.

Allogeneic Peripheral Blood Stem Cell Transplantation (PBSC)

On day 0, the patient received the cryopreserved PBSC (prepared as described above). The cryopreserved PBSC product was thawed and administered intravenously immediately. The target dose of the PBSC was ≧4×10⁶ CD34⁺ cells per kg. However, if donor apheresis on days 5, 6, and 7 yielded a total of ≧3×10⁶ CD34⁺ cells per kg, this level of CD34⁺ cell dose was utilized.

Growth Factor Administration Post-transplant

On day 0 of the transplant, immediately after PBSC transfusion, patients begin treatment with recombinant human filgrastim at a dose of 10 μg/kg/day s.c. Filgrastim continues until the ANC count is greater than 5000 cells per μl for three consecutive days.

### EXAMPLE 8

### Depleting a Subject's Immune System Prior to Administration of Th2 Cells

Prior to transplantation of Th2 cells, the transplant recipient's immune system is depleted or ablated using any immune-depleting method. Specific examples are provided below, although other methods can be used.

Until recently, preparative regimens utilized for allogeneic bone marrow transplantation (alloBMT) have generally included myeloablative doses of chemotherapy and/or total body irradiation. The high level of leukemia relapse that occurs in the setting of T cell-depleted alloBMT indicates that the curative anti-leukemic aspect of marrow transplantation is likely derived primarily from a T cell-mediated GVL effect, and not from the myeloablative preparative regimen. These inadequacies of myeloablative preparative regimens, combined with the high levels of morbidity and mortality associated with myeloablation, results in a low therapeutic index for this aspect of allogeneic transplantation. As such, the ability to establish alloengraftment without myeloablation is disclosed.

Fludarabine can contribute to the prevention of marrow rejection in non-myeloablative transplant approaches. In the setting of T cell-replete HLA-matched PBSCT, fludarabine-based preparative regimens have consistently resulted in donor engraftment without myeloablation. A Phase III study of Th2 cells for the prevention of GVHD after non-myeloablative allogeneic PBSCT will be performed. It is thought that administration of donor CD4⁺ Th2 cells will allow for donor engraftment after fludarabine-based regimens with reduced GVHD.

Immune-Depleting Chemotherapies

Subjects received at least one cycle of induction chemotherapy, even if their CD4⁺ count was less than 50 cells per μl. If the subject is also the donor of the Th2 cells, chemotherapy is not administered until after cell products are harvested from the subject. Placement of permanent central venous access can be performed. Ideally, steroids are not used as an anti-emetic during this chemotherapy regimen. Examples of immune depleting chemotherapy that can be used to deplete a subject's immune system prior to Th2 cell therapy include the Fludarabine/EPOCH method (Table 3) and the Fludarabine/cyclophosphamide method (fludarabine (25 mg/m² per day IV for 4 consecutive days) combined with cyclophosphamide (600 mg/m² per day IV for 4 days)). However, other methods known to those skilled in the art may also be employed.

TABLE 3
Cycle 1 of Induction Chemotherapy

| Drug | Dose | Days |
|---|---|---|
| Fludarabine | 25 mg/m² per day IV | Days 1, 2, 3 |
| | Infusion over 30 minutes, | |
| | daily for 3 days | |
| Etoposide | 50 mg/m² per day continuous | Days 1, 2, 3 |
| | IV Infusion over 24 hours, | |
| | daily for 3 days | |
| Doxorubicin | 10 mg/m² per day continuous | Days 1, 2, 3 |
| | IV Infusion over 24 hours, | |
| | daily for 3 days | |
| Vincristine | 0.5 mg/m² per day continuous | Days 1, 2, 3 |
| | IV Infusion over 24 hours, | |
| | daily for 3 days | |
| Cyclo- | 600 mg/m² IV Infusion over | Day 4 |
| phosphamide | 2 hr | |
| Prednisone | 60 mg/m² per day orally, | Days 1, 2, 3, 4 |
| | daily for 4 days | |
| Filgrastim | 10 μg/kg per day | Daily from day 5 |
| | subcutaneously | until ANC > |
| | | 1000/μl for two |
| | | consecutive days |

Fludarabine can be obtained from Berlex Laboratories (FLUDARA IV). Cyclophosphamide is available from Mead Johnson (Cytoxan). Etoposide/Doxorubicin/Vincristine can be administered as a continuous infusion (all are commercially available). In this study, the daily dose of vincristine, doxorubicin, and etoposide (i.e., the 24 hour supply) is admixed together in 500 ml of 0.9% NaCl injection and delivered with a suitable infusion pump through a central venous access device. The bag is exchanged daily for each of the three days to complete the 72 hour infusion It is noted that doxorubicin cardiotoxicity is particularly noted after cumulative doses of greater than 550 mg/m². Prednisone is commercially available in solid or liquid dosage forms. In patients unable to tolerate oral medication, methylprednisolone is substituted at the same dosage, diluted in 25 ml of D5W, and infused over 15 minutes. Ideally, prednisone should be taken with food to reduce gastrointestinal side effects. Filgrastim (G-CSF) is a commercially available recombinant human protein (Neupogen; Amgen Corp., Thousand Oak, Calif,). Filgrastim should not be diluted with NaCl solutions.

Because the primary purpose of the induction chemotherapy is to establish severe host immune T cell depletion prior to the allotransplant, the number of induction chemotherapy cycles administered was determined by the severity of immune T cell depletion observed. The CD4⁺ count can be measured by flow cytometry, for example in the interval of day 15 to day 21 of the fludarabine/EPOCH chemotherapy. If there were >50 CD4⁺ cells per μl of blood during this interval, further cycles of induction chemotherapy were administered (in an attempt to achieve greater immunosuppression prior to transplantation). However, a maximum of three cycles of induction chemotherapy were administered. If the level of CD4⁺ cells is <50 cells per μl of blood when measured within days 15-21 after fludarabine/EPOCH administration, this indicated that the immune system of the subject is adequately depleted, and that subject received the transplant preparative regimen.

Subjects received the second cycle of chemotherapy on day 22 after the first cycle was initiated. However, an additional two weeks of recovery time before administration of the second cycle was provided if medically indicated (for example, for delay in neutrophil recovery, documented infection, or other complication resulting from the induction chemotherapy regimen).

If a subject developed neutropenia of less than 500 PMN's per ill for more than seven days during any cycle of induction chemotherapy, the subject received no further induction chemotherapy. Instead, they received a transplant preparative regimen (see below), even if the CD4⁺ count was not <50 cells per μl.

Following chemotherapy, subjects proceeded to the transplant preparative regimen chemotherapy (even if the CD4⁺ count is still >50 cells per μl). If a subject developed progressive disease at any point during induction chemotherapy cycles, such a subject proceeded to the transplant preparative regimen (independent of the CD4⁺ count).

Determination of Cycle 2 and Cycle 3 Dose Escalation

If the first cycle of induction chemotherapy does not reduce the CD4⁺ count to below 50 cells per μl and does not result in febrile neutropenia or prolonged neutropenia as evidenced by two consecutive bi-weekly ANC values less than 500 cells per μl, then the next cycle of induction chemotherapy can be dose escalated, by increasing the daily dose of fludarabine, etoposide, adriamycin, and cyclophosphamide 20%. If a third cycle of chemotherapy is required (CD4⁺ count still greater than 50) and febrile neutropenia or two timepoints of ANC less than 500 did not occur after cycle 2, then the third cycle of induction chemotherapy is administered at a further 20% escalation of doses administered for cycle 2.

Dose Reduction of Pre-transplant Induction Chemotherapy

In the event that more than one subject experiences a period of neutropenia (ANC less than 500 per μl) for more than 10 days, the etoposide, doxorubicin, vincristine, and prednisone is reduced from three days to two days of administration. The doses of these medications remain unchanged. In the event of this change, the cyclophosphamide and filgrastim is given on day 3. The same schedule modification described in subsection a) (above) is performed if any grade IV toxicity by the NCI Common Toxicity Criteria is observed in more than one subject.

Transplant Preparative Regimen

On day 22 after the final cycle of induction chemotherapy, subjects were eligible to receive a transplant preparative regimen (Table 4). Therefore, day 22 of the final induction chemotherapy cycle is transplant day −6. However, in cases where additional recovery time was required (for example, due to prolonged neutropenia, documented infection, or other medical complications of the induction regimen), an additional two weeks of recovery time was utilized prior to initiation of the transplant preparative regimen.

TABLE 4
Transplant Preparative Regimen

| Drug | Dose | Days |
|---|---|---|
| Fludarabine | 30 mg/m² per day IV | Transplant |
| | Infusion over 15 to 30 | Days −6, −5, −4, −3 |
| | minutes, daily for 4 days | |
| Cyclo- | 1200 mg/m² per day IV | Transplant |
| phosphamide | Infusion over 2 hours, | Days −6, −5, −4, −3 |
| | daily for 4 days | |
| Mesna | 1200 mg/m² per day by cont- | Transplant |
| | inuous IV Infusion, | Days −6, −5, −4, −3 |
| | daily for 4 days (start | |
| | 1 hour before | |
| | cyclophosphamide)* | |

*Bag #1 of the mesna is 150 mg/m² in 250 ml over a 3 hour infusion (thus stopping when cyclophosphamide ends). Then, mesna is given at 1200 mg/m² in 500 ml over 24 hour infusion, for four days (days −6, −5, −4, and −3). Mesna (sodium 2-mercaptoethanesulfonate) is commercially available as Mesnex (Asta Medica).

Hydration Regimen during Preparative Regimen Chemotherapy

Hydration was initiated 12 hours prior to cyclophosphamnide infusion (on day −7 of the transplant). Hydration was with normal saline supplemented with 10 meq/liter KCl at a rate of 100 ml/hour. Hydration continued until 24 hours after the last cyclophoshamide dose was completed. During hydration, 20 mg of furosemide was administered daily by IV route to maintain diuresis. If body weight in any patient increased to more than 5% above pre-cyclophosphamide weight, additional doses of furosemide were administered. In general, furosemide doses are separated by at least a four hour observation interval. During hydration, serum potassium levels were monitored every 12 hours. If potassium value was >4.5 meq/1, KCl was removed from the saline infusion. If potassium value was <3.0, KCl concentration in the saline is increased to 25 meq/1. During hydration, if urine output was <1.5 ml/kg/hour, an additional 20 mg of furosemide was administered.

Monoclonal Antibody Therapies

Examples of monoclonal antibody therapies that can be used to practice the disclosed methods include, but are not limited to: Rituxan and Herceptin. Rituxan is a monoclonal antibody to CD20, which is present on B cell malignancies such as lymphoma. Herceptin is a monoclonal antibody to her2-neu, which is often over-expressed on breast cancer cells. These agents are typically administered in combination with chemotherapy. In general, monoclonal-antibody based therapy is well-tolerated so a high degree of monitoring is not required.

### EXAMPLE 9

### Infection Prophylaxis

To assist in protecting a subject from infections that can result from receiving chemotherapy or other immune-depleting therapy, one or more prophylactic compounds can be administered prior to the start of the therapy, to enhance the immune system. The prophylaxis disclosed below can be administered separately, or in combination, depending on the requirements of the subject. In addition, the dosage regimens for the prophylaxis described below are known to those skilled in the art, and can be found in Mandell (Principles and Practice of Infectious Disease; 5th Edition, Copyright 2000 by Churchill Livingstone, Inc.)

For example, at the initiation of pre-transplant induction chemotherapy until administration of immunosuppressive agents is terminated, subjects can receive: trimethoprim 160 mg/sulfamethoxazole 800 mg for PCP prophylaxis, one tablet p.o. BID on two days of every week (i.e., on each Saturday and Sunday). If a subject is allergic to sulfonamide antibiotics, aerosolized pentamadine (300 mg) is administered at the time of transplant regimen chemotherapy administration, and then once per month until the patient is off of immunosuppressive agents. After completion of this one week treatment period with trimethoprimi sulfamethoxazole, this drug is not administered until the absolute neutrophil count reaches 1000 cells/μl and the platelet count reaches 50,000 cells/μl after the allogeneic PBSCT. When the ANC reaches 1000 cells/μl and the platelet count reaches 50,000 cells/μl, the subject will resume an oral regimen of trimethoprim 160 mg/sulfamethoxazole 800 mg, BID on two days per week. This regimen will continue until the patient is off of immunosuppressive agents.

Acyclovir (800 mg p.o. BID or 250 mg/m² i.v. every 12 hours) can be administered for HSV prophylaxis.

In addition, fluconazole can be administered (400 mg p.o. daily, oral or i.v.) for fungal and bacterial prophylaxis. However, because fluconazole can delay the clearance of vincristine, fluconazole is discontinued during days 1, 2, 3, and 4 of induction chemotherapy. Fluconazole is then restarted on day 5 of the cycle (along with the G-CSF initiation). In the case of either an ANC less than 500 cells/μl and any fever in excess of 38.0° C., a third or fourth generation cephalosporin is initiated.

In addition, IVIG (500 mg/kg IV) starting at day 28 post-transplant, and continuing every two weeks until day 100 post-transplant, can be administered for CMV prophylaxis and treatment (Table 5). At that point, IVIG is reduced to 500 mg/kg every four weeks until day 180.

TABLE 5
IVIG Administration (500 mg/kg IV)

| | |
|---|---|
| 4 | Weeks post-transplant |
| 6 | Weeks post-transplant |
| 8 | Weeks post-transplant |
| 10 | Weeks post-transplant |
| 12 | Weeks post-transplant |
| 14 | Weeks post-transplant |
| 18 | Weeks post-transplant |
| 22 | Weeks post-transplant |
| 26 | Weeks post-transplant |

After day 180, further IVIG administration is dependent on serum immunoglobulin levels and degree of immunosuppression. If CMV infection is documented, acyclovir is discontinued and the patient starts on ganciclovir, 5 mg/kg i.v. every 12 hours for 14 consecutive days. Ganciclovir is then maintained at a dose of 5 mg/kg i.v. daily until the patient is off of immunosuppressive agents. The dosage and schedule of ganciclovir is modified for renal insufficiency. During ganciclovir treatment of established CMV infection, IVIG is administered at a dose of 500 mg/kg i.v. each week.

### EXAMPLE 10

### GVHD Chemoprophylaxis with Cyclosporine A

In one example, cyclosporine (CSA; available as an injectable concentrate (Sandimmune) or as a microemulsion in capsules (Neoral)) is initiated on the day −1 before the transplant. CSA is administered by i.v. infusion at a dose of 2 mg/kg. CSA is administered every 12 hours, with each infusion administered over a 2 hour period. In the first two weeks post-transplant, CSA dose is modified to achieve adequate steady-state CSA levels. Once this intravenous dose is established and the patient is able to tolerate oral feedings (typically by day 14 post-transplant), then CSA is switched to the oral formulation. Conversion of CSA to the oral formulation is typically performed by multiplying the adequate i.v. dose by a factor of 1.5 to 2.0. Patients are then maintained on oral CSA on a 12 hour schedule. This dose of CSA continues until day 100 post-transplant, at which point it is gradually tapered as long as the level of GVHD is less than grade 2 (Table 6). Taper consists of a 5 to 10% dose reduction each week (patient is then taken off of CSA by day 180 post-transplant). Blood or plasma concentrations of CSA are typically monitored; concentrations of 250 ng/ml (blood) or 50 ng/ml (serum) appear to minimize the frequency of CSA adverse effects.

TABLE 6
GVHD Chemoprophylaxis With Cyclosporine A

| | | CSA Dosage |
|---|---|---|
| Taper Step | Days post-BMT | (mg/kg/dose) |
| Taper Step 1 | 101-107 | 95% of Maintenance |
| | | Dose (M.D.) |
| Taper Step 2 | 108-114 | 90% of M.D. |
| Taper Step 3 | 115-121 | 85% of M.D. |
| Taper Step 4 | 122-128 | 80% of M.D. |
| Taper Step 5 | 129-135 | 70% of M.D. |
| Taper Step 6 | 136-142 | 60% of M.D. |
| Taper Step 7 | 143-149 | 50% of M.D. |
| Taper Step 8 | 150-156 | 40% of M.D. |
| Taper Step 8 | 157-163 | 30% of M.D. |
| Taper Step 10 | 164-170 | 20% of M.D. |
| Taper Step 11 | 171-180 | 10% of M.D. |

Ideally, the decision to taper CSA before day 100 is permitted only if clinically indicated. For example, taper of CSA before day 100 is permitted for the treatment of clinically evident progressive disease post-transplant, and for the treatment of low levels of donor chimerism post-transplant (less than 20% donor chimerism by day 60 post-transplant).

In one example, rapamycin can be used instead of, or in addition to, CSA, for GVHD prophylaxis. For example a loading dose of oral rapamycin of 15 mg per meter squared of body surface area, followed by a maintenance dose of 5 mg per meter squared per day orally for the next 13 days, can be administered (for example see Benito et al., Transplantation, 72(12):1924-29, 2001).

### EXAMPLE 11

### Treatment of Persistent Disease Post-Transplant

DLI and Other Therapy

Subjects with persistent or progressive malignant disease post transplant, such as 100 days after an allogeneic stem cell transplant, is a poor prognostic sign. When relapse occurs after transplantation, administration of additional donor immune cells, such as donor Th2 cells, at the time of relapse can result in tumor regressions. This form of immune therapy, because it occurs at a time remote from the original stem cell transplant procedure, is termed “delayed donor lymphocyte infusion” (DLI). DLI may be 20 administered alone or after chemotherapy administration.

Donor lymphocytes are collected by apheresis either in steady state (no donor therapy) or after G-CSF mobilization. The donor product can be enriched for lymphocytes by Ficoll-Hypaque procedure to a buffy coat product Alternatively, in cases where additional donor stem cells are desired, the donor product can be administered without lymphocyte purification. DLI can be sequentially administered, with initial dosing at 1×10⁶ CD3⁺ T cells per kg, and with subsequent dose increases to 1×10⁷ and 1×10 ⁸ per kg.

Allogeneic Th2 lymphocytes have an application in improving the results of DLI therapy for the treatment of malignancy post-transplant. In this Th2-modified DLI method, a subject suffering from a malignant relapse following an allogenic stem cell transplant is immuno-depleted, such as using chemotherapy as described in EXAMPLE 8, to deplete or eliminate an immune system that is not efficient in eliminating the cancer. In one example, immune-depleting chemotherapy includes fludarabine administration followed by EPOCH chemotherapy, with subsequent administration of fludarabine and higher doses of cyclophosphamide. After immune depletion, the subject is administered additional donor CD4⁺ and CD8⁺ T cells in the dose range of 40 to 400×10⁶ T cells per kg. Within 24 hours after this T cell administration, the subject additionally receives the ex vivo generated donor CD4⁺ Th2 cells, for example between 5 and 125×10⁶ cells/kg, using the methods described in Examples 1-3. This method results in a more potent DLI approach with respect to increased anti-tumor efficacy. Additionally, because the Th2 infusion will moderate GVHD, this Th2 DLI method mediates anti-tumor effects with reduced GVHD-related toxicity.

Alternatively, persistent or progressive disease can be treated with any approved therapy thought to be in the best standard care of the patient, such as chemotherapy, cytokine therapy, or monoclonal antibody therapy. Alternatively, patients with relapse may receive therapy on other NCI protocols.

Treatment of Graft-Versus-Host Disease

In patients where GVHD is suspected, standard clinical criteria and skin or liver biopsy information is used to establish the diagnosis. Acute GVHD is graded by the Glucksberg criteria (Table 7). Subjects with clinical stage 1 or 2 of the skin without any other organ involvement are treated with a 1% hydrocortisone creme BID. In general, patients with ≧Grade II acute GVHD are treated with high-dose corticosteroids. Patients who fail to respond satisfactorily to corticosteroids can receive anti-thymocyte globulin (ATG) treatment or other experimental acute GVHD protocols.

TABLE 7
Four Point Grading Scales for GVHD
Target Organs (Glucksberg Criteria)

| Skin | Liver | Intestine |
|---|---|---|
| 1 of 4: Rash | 1 of 4: Bilirubin | 1 of 4: Diarrhea |
| on <25% BSA | of 2-3 mg % | of >500 ml/day |
| 2 of 4: Rash | 2 of 4: Bilirubin | 2 of 4: Diarrhea |
| on 25 to 50% BSA | of 3.1-6 mg % | of >1000 ml/day |
| 3 of 4: Rash | 3 of 4: Bilirubin | 3 of 4: Diarrhea |
| on >50% BSA | of 6.1-15 mg % | of >1500 ml/day |
| 4 of 4: Bullae | 4 of 4: Bilirubin | 4 of 4: Pain |
| Desquamation | of >15 mg % | and Ileus |

TABLE 8
Clinical Grading of Acute GVHD (highest score
during 100 day post-transplant period)

| Clinical Grade of | Target Organs of GVHD | | |
|---|---|---|---|
| Acute GVHD | Skin | Liver | Intestine |
| Grade 0 | None | None | None |
| Grade I | 1 or 2 | None | None |
| Grade II (SLI) | 1, 2, or 3 | 1 | 1 |
| Grade II (S) | 4 | None | None |
| Grade II (LI) | None | 1 | 1 |
| Grade III | 2 or 3 | 2 or 3 | 2 or 3 |
| Grade IV | 2, 3, or 4 | 2, 3, or 4 | 2, 3, or 4 |

Acute GVHD can be treated as follows. Grade 0-I GVHD is treated with topical corticosteroids (1% hydrocortisone or equivalent) applied to rash BID. Grade H-IV GVHD is treated with methylprednisolone (MP) 62.5 mg/m² per dose IV, BID for 4 consecutive days. If there is no response after 4 days, continue until response (7 day maximum trial). If response within 7 days, taper as follows: 50 mg/m² per dose IV, BID for 2 days; 37.5 mg/m² per dose IV, BID for 2 days; 25 mg/m² per dose IV, BID for 2 days; 10 mg/m² per dose IV, BID for 2 days; after this, steroid will be reduced by 10% each week. During taper, maintain CSA at therapeutic levels (trough level should be greater than 150 ng/ml). When clinically appropriate, change MP to the potency equivalent of oral prednisone (10 mg dose of MP is as potent as 12.5 mg of prednisone). If GVHD worsens during taper, steroids can be increased to previous dose. If there is no response observed within 7 days of MP treatment, methylprednisolone can be increased to 500 mg/m² per dose IV, BID for 2 days. If there is no improvement, steroids are discontinued, and consideration will be made of using other agents for the treatment of GVHD (such as anti-thymocyte globulin or other more experimental options that may be available). Ideally, during steroid treatment of GVHD, fluconazole is changed to itraconazole.

The following are criteria to determine definitions of response to acute GVHD treatment. Determination of acute GVHD treatment response should be made within 96 hours of starting the treatment Complete response: complete resolution of all clinical signs and symptoms of acute GVHD. Partial Response: 50% reduction in skin rash, stool volume or frequency, and/or total bilirubin; maintenance of adequate performance status (Karnofsky Score ≧70%, see Table 2). Non-responder: <50% reduction in skin rash, stool volume or frequency, and/or total bilinibin; failure to maintain adequate performance status (Karnofsky Score ≦70%, see Table 2). Progressive disease: fruter progression of signs and symptoms of acute GVHD, and/or decline in performance status after the initiation of therapy.

Chronic GVHD can be treated as follows. Initial therapy involves 7 days of treatment with the following regimen: cyclosporine, 6 mg/m² per dose orally BID and prednisone, 1 mg/kg per dose, orally once daily. If response is observed after 7 days, the following taper is performed: prednisone, 1 mg/kg per dose given every other day (no day 8 dose); CSA, 6 mg/m² per dose given BID every other day (no day 9 dose); CSA and prednisone dosing are thus given on alternate days. Further taper is by decreasing prednisone by 0.1 mg/kg per dose each week; patient will thus be off of prednisone in 10 weeks. During this prednisone taper, the patient will be maintained on the same dose and schedule of CSA. CSA is then given on the following monthly taper: month 1, CSA 4.5 mg/m² per dose, given BID every other day; month 2, CSA 3.0 mg/m² per dose, given BID every other day; month 3, CSA 1.5 mg/m² per dose, given BID every other day; then discontinuation of CSA. If no response is observed after 7 days, continue an additional seven days or until response, whichever comes first After response, taper as above.

If no response is observed after 14 days of initial CSA/prednisone treatment these agents are discontinued, and consideration is made towards alternative treatments of chronic GVHD such as: immuran or mycophenolic acid; ATG; or other experimental treatments.

The following are criteria to determine definitions of response to chronic GVHD treatment. As with acute GVHD, determination of chronic GVHD response to treatment should be made within 96 hours of treatment initiation. Complete response: complete resolution of all clinical signs and symptoms of chronic GVHD. Partial response: Clinical improvement, but persistence in signs and symptoms of chronic GVHD; maintenance of adequate performance status (Karnofsky Score ≧70%, see Table 2). Stable disease: No improvement or progression in signs and symptoms of chronic GVHD; maintenance of adequate performance status (Karnofsky Score ≦70%). Non-responder: progression in signs and symptoms of chronic GVHD, and/or decline in performance status after initiation of treatment.

### EXAMPLE 12

### Pharmacokinetic and Immune Studies

The methods below describe how subjects can be monitored before, during, and after treatment.

Evaluation of Pre-Transplant Induction Chemotzerapy Cycles

Blood samples (10 cc in green-top heparinized tube) are drawn to evaluate the effects of immune depletion. This sample is drawn just prior to each cycle of induction chemotherapy (within six days of the next cycle). Experiments can include the use of flow cytometry to detect depletion of lymphoid versus myeloid subpopulations during induction chemotherapy.

Evaluation of Transplant Chemotherapy Preparative Regimen

Blood samples (10 cc in green-top hepariinized tube) are drawn to evaluate the effects of the fludarabine and cyclophosphamide regimen on immune depletion in a subject. Timepoints that can be used are: 1) immediately prior to preparative regimen chemotherapy (day −6); and 2) just prior to the PBSCT (day 0). Experiments consist of flow cytometry to detect depletion of host lymphoid versus myeloid subpopulations in the peri-transplant period.

Cyclosporine Monitoring

Blood samples (5 cc in green-top heparinized tube) are tested twice per week in the first two weeks post-transplant, and then once per week for the next two weeks. After the first four weeks post-transplant, additional blood for CSA levels is sent if clinically indicated (occurrence of nausea, vomiting, headaches, hypertension, increased creatinine).

Evaluation of Type I Versus Type H Cytokine Effects Post-transplant

Blood samples (40 cc in green-top heparinized tubes, and 10 cc in serum collection tubes) are drawn once weekly at the following timepoints: prior to starting induction chemotherapy, prior to each induction chemotherapy cycle, and then each week after transplant administration for the first 100 days post-transplant. Samples are analyzed to measure plasma levels, intracellular cytokine levels, and gene expression analysis of type I versus type II cytokines in the first 100 days post-transplant, with correlations being made to level of GVHD observed. If there is a clinically-significant increase in the level of GVHD, blood samples can be drawn to test for cytokine changes during GVHD.

Determination of Donor/Host Chimerism Post-Transplant

Blood samples (10 cc in green-top heparinized tube) are drawn to evaluate the extent of donor versus host chimerism post-transplant. If a result of mixed chimerism is obtained at day 15 post-transplant, subsequent draws are increased to 60 ml of blood so that cell sorting experiments can be performed (to evaluate chimerism in cell subsets). Timepoints for chimerism analysis are: day 15, day 30, day 60, and day 100 post-transplant. After day 100, chimerism is determined if clinically indicated (in the setting of disease relapse). Chimerism can be evaluated using a PCR-based assay.

Evaluation of Immune Reconstitution Post-Transplant

Blood (25 ml in heparinized tube) is evaluated for immune reconstitution post-transplant Included is an evaluation of T cell receptor diversity post-transplant using a PCR-based assay. Samples are evaluated monthly for 3 months, and then every 3 months for the first two years post-transplant.

On Study Evaluation

Clinical blood tests (CBC with differential, electrolytes, liver and mineral panels): for induction chemotherapy period, day 1 and then twice per week; for inpatient period post-transplantation, daily; after discharge post-transplant, once per week. Follow-up visits are at day 140, day 180, day 290, and day 365 post-transplant Patients are followed every six months for one year, and then yearly until 5 years post-transplant.

Off Study Criteria

Patients are removed from the clinical trial if there is irreversible dose limiting toxicity during the induction chemotherapy cycles. This is defined as any grade IV toxicity which precludes the patient from receiving the chemotherapy on the timeline detailed in the study. In addition, patient non-compliance or patient withdrawal can be grounds for removal of the subject from the clinical trial.

Post-Study Evaluation

Clinic visits are continued for a period of two years post-transplant. Management of patients during this period with regard to blood tests and radiographic studies are performed as clinically indicated.

Toxicity Criteria

The NCI Common Toxicity Criteria version 2.0 is used. This document can be found at the NIH website. For this study, the development of hyperacute GVHD is considered a toxicity likely attributable to the Th2 cell administration. Hyperacute GVHD is defined as severe GVHD (grade 3 or 4) that occurs in the first 14 days post-transplant.

### EXAMPLE 13

### Clinical Trial Results

Immunoablative Induction and Preparative Regimen

Disclosed herein is a novel reduced intensity allogeneic PBSCT protocol that incorporates host immune T cell ablation prior to PBSCT. An induction chemotherapy regimen of fludarabine in combination with the agents contained in the EPOCH regimen (see Example 8) were used. The primary purpose of administering this chemotherapy cycle was to achieve a high-level of host immunosuppression prior to allotransplantation. The development of induction chemotherapy regimens which induce severe host T cell depletion, without myeloablation, is a desirable goal. Attempts have been made to reduce the CD4 count to less than 50 cells per μl prior to administration of the transplant preparative regimen. This level of host CD4⁺ T cell depletion is associated with significant immunosuppression and a reduced ability to reject allogeneic cells in patients with 13 cell malignancy.

An initial treatment cohort not receiving donor Th2 cells (n=19) was studied to evaluate an immunoablative reduced intensity regimen, and to determine the incidences of mixed chimerism, GVL effects, and acute GVHD associated with this method. Patients, most of whom had chemotherapy-refractory non-Hodgkin's lymphoma, were treated with outpatient EPOCH combined with fludarabine (EPOCH-F; 1 to 3 cycles, see Example 8) to reduce malignancy and reducie host immune T cell numbers prior to PBSCT. In each case, a marked reduction in T cells and either stable disease or partial disease responses to the chemotherapy was observed. EPOCH-F reduced median host CD4⁺ and CD8⁺ T cells from 239 to 63 cells/μl and from 242 to 62 cells/μl, respectively.

Following fludarabine and EPOCH induction chemotherapy, patients received preparative regimen chemotherapy (concomitant fludarabine [30 mg/m²/day×4] and cyclophosphamide [1200 mg/m²/day×4]). This treatment further reduced host CD4 and CD8 counts to median values of 2.3 and 0.4 cells/μl, respectively. This immunoablative host preparation resulted in rapid complete donor engraftment, with median day 14 post-SCT donor lymphoid and myeloid chimerism of 98 and 99%, respectively. Rapid engraftment was associated with clinically significant anti-tumor responses, which occurred without donor lymphocyte infusion or removal of GVHD prophylaxis (single-agent cyclosporine A). A significant component of the anti-tumor effect was likely due to an allogeneic GVL effect, as the complete response rate advanced from 7/19 (36.8%) at day 28 post-SCT to 12/18 (66.7%) at day 100 post-SCT. However, this GVL effect was associated with acute GVHD (range of grade I to grade m GVHD severity, on the Glucksberg scale of 0 to IV) of Grade 0 to I (7/19), Grade II (6/19), or Grade III (6/19), which occurred at a median of 31 days post-SCT. Five patients had a GVHD severity that necessitated the institution of systemic steroids in addition to the standard CSA GVHD prophylaxis. Therefore, host immunoablation prior to reduced intensity allogeneic PBSCT results in rapid donor engraftment and allogeneic GVL effects, but is limited by acute GVHD. Because of this significant rate and severity of GVHD, the use of Th2 cells in this allogeneic PBSCT setting was examined.

Allogeneic PBSCT in the Treatment of Leukemia and Lymphoid Neoplasia

Allogeneic bone marrow transplantation represents a potential treatment for patients with multiple hematologic and lymphoid malignancies. The allogeneic GVL effect contributes to disease remission in acute lymphocytic leukemia, acute myelogenous leukemia, chronic lymphocytic leukemia, chronic myelogenous leukemia, indolent and high-grade non-Hodgkin's lymphoma, Hodgkin's lymphoma, multiple myeloma, and myelodysplastic syndrome. Because the EPOCH regimen has an established response rate in patients with chemotherapy-refractory lymphoid malignancy, such patients were eligible for this study. The addition of fludarabine to EPOCH may further improve the anti-tumor effects of this regimen. However, the activity of fludarabine and EPOCH chemotherapy in patients with leukemia is not known. As such, patients with leukemia (AML, myelodysplasia, ALL, and CL) were candidates for this protocol only if they had a relatively low disease burden (<10% blasts).

Allogeneic SCT with Th2 Cells: Initial Phase I Results

Using the immunoablative reduced intensity regimen described above, the additional use of donor CD4⁺ Th2 cells after G-CSF mobilized, T cell replete allogeneic PBSCT was examined.

Donor CD4 cells were cultured ex vivo as described in the above Examples to enhance Th2 differentiation. In all cases, the culture method disclosed herein generated human donor Th2 cells that were 68%-99% pure for CD4^{'} T cells and less than 1% contaminated by CD8⁺ T cells. Cultured Th2 cells were administered on the day after allogeneic stem cell transplantation.

The initial three patients were enrolled to Th2 cell dose level #1 (5×10⁶ Th2 cells/kg). Acute GVHD grade II (n=2) and grade m (n=1) were observed; although, no serious adverse events attributable to the Th2 cells were identified. Since no hyperacute GVHD or grade 4 or 5 toxicity attributable to the Th2 cells was observed, and there was no apparent decrease in acute GVHD with this Th2 dose, it was determined that this dose level was safe, and accrual to dose level #2 commenced.

In the second Th2 dose cohort (25×10⁶ cells/kg; n=6), the initial patient entered a pathologic complete remission from refractory bulky lymphoma, but died of DIC and shock at day 22 post-SCT (grade I clinical GVHD). Subsequent patients at Th2 level #2 had rapid recovery of hematopoiesis, with full donor lymphoid and myeloid chimerism without significant toxicity. That is, in each case, the total blood mononuclear cell donor chimerism, as indicated by VNTR PCR analysis, was at least 99% by day 14 post-transplant (range 99 to 100%). Therefore, the Th2 cells do not appear to impair or negatively influence engraftment By comparison to other published studies using allogeneic stem cell transplantation preparative chemotherapy regimens similar to that used herein (preparative regimens of reduced intensity; “non-myeloablative”), it appears that this high level of donor chimerism early post-transplant has not been previously documented. As such, allogeneic transplantation in this manner with Th2 cells results in very rapid donor engraftment. Because it is well established that optimal anti-tumor effects occur when full donor chimerism is established post-transplant, this clinical result is advantageous.

In all cases, Th2 cell administration resulted in immune cell activation post-transplant. One characteristic of this immune activation was early alloengraftment which is initially primarily of lymphoid origin In traditional allogeneic transplantation, engraftment is heralded by a return of myeloid cell populations post-transplant However, in the Th2 recipients, lymphoid cell populations predominated early post-transplant (at day 7 to 10), followed by stable myeloid engraftment. Therefore, Th2 cell administration results in an immune activation characterized by early return of lymphocyte populations post-transplant Flow cytometry studies characterized this early donor lymphocyte engraftment as including of both CD4^{'} and CD8⁺ T cells. Because the Th2 cell infusion contains only CD4⁺ T cells, the Th2 cell infusion activates both CD4⁺ and CD8⁺ lymphoid T cells present in the G-CSF-mobilized peripheral blood stem cell graft. Thus, without being bound by theory, the Th2 cells likely activate the immune cells contained in the conventional allogeneic stem cell transplant procedure.

Four cases were tested to determine the cytokine secretion cells of lymphocytes harvested from recipients. Immune T cells harvested from Th2 recipients at day 12 to 14 post-transplant were capable of secreting both type I (IFN-γ) and type II (IL-4, IL-10) cytokines. This cytokine-secreting ability has not been demonstrated in other non-Th2 cell transplant recipients. Thus, this provides further evidence that the Th2 cells augment immune cell activation post-transplant. Cytokine secretion post-transplant in Th2 recipients appears to be augmented in both CD4⁺ and CD8⁺ T cells. Without being bound by theory, this result is consistent with a Th2 cell effect that results in immune cell activation of both CD4 and CD8 cells contained in the allogeneic stem cell graft. Th2 cell activation of both type I and type II cytokines post-transplant is likely to be advantageous relative to sole activation of only type II cytokines (which may be associated with reduced anti-tumor effects).

At dose level #2 of Th2 cells (25×10⁶ cells/kg), there appeared to be reduced GVHD, with four patients having no clinical acute GVHD and one having only acute GVHD grade III (liver). Anti-tumor responses were observed in refractory malignancy patients, including a molecular CR in a patient with accelerated phase CML. In each Th2 recipient that was evaluable for malignant disease response, at some point in the post-transplant course, a reduction in tumor burden was observed. Thus, without being bound by theory, Th2 cell administration appears to initiate an immune cell activation, and that this activation is associated with the observed anti-tumor effects post-transplant.

Because Th2 dose level #2 achieved alloengraftment with anti-tumor responses (and therefore augmentation of T cell replete allografts with co-stimulated Th2 cells does not appear to abrogate allogeneic GVL effects) and limited GVHD (2/6 grade I-IV acute GVHD), this amount was a candidate for evaluation in the phase II aspect of the protocol.

Allogeneic SCT with Th2 Cells: Phase II

Current study accrual is proceeding on Th2 level #3 (125×10⁶ cells/kg; n=6). If the safety and feasibility of dose level #3 is demonstrated in these initial six subjects, 18 additional subjects will be treated with Th2 cells at dose level #3 (125×10⁶ cells/kg). If dose level #3 results in more than 1/6 Th2-related adverse events or more than 2/6 cases of grade f to IV acute GVHD, the additional 18 subjects will be treated at Th2 cell dose level #2 (25×10⁶ cells/kg). If recipients of Th2 dose level #3 have 0/6 or 1/6 cases of severe toxicity and 0/6, 1/6, or 2/6 cases of grade II to IV acute GVHD, the additional 18 patients will be treated at dose level #3. If the high dose of Th2 cells can not be consistently generated, the phase II component of accrual may be initiated at dose level #2.

Therefore, 24 total patients will be treated with a defined dose of Th2 cells, either 25 or 125×10⁶/kg. The rate and severity of acute GVHD in these 24 Th2 recipients will be compared to the initial protocol cohort of 19 patients receiving transplantation without Th2 cells (12/19 with grade II to m acute GVHD). This study allows one to determine whether Th2 administration reduces acute GVHD relative to the initial cohort that did not receive Th2 cells.

It is proposed that recipients of the Th2 cells will have reduced GVHD relative to non-Th2 recipients. In the cohort of non-Th2 recipients, the incidence of grade II to grade IV acute GVHD was 12/19. Therefore, the true rate of grade II to IV GVHD without Th2 cells is approximately 60%. IThe expanded cohort of n=24 Th2 recipients may have a significantly reduced incidence of grade I to IV acute GVHD. For example, the incidence of grade II to IV acute GVHD may be reduced from 60% without Th2 cells to 20% with Th2 cells.

The predicted power to detect a Th2-mediated reduction in grade II to IV acute GVHD from 60% to 20% in the expanded Th2 cohort will depend on the incidence of grade II to IV GVHD observed during the phase I trial. Using a two-tailed conditional power statistical analysis at the p=0.05 level accrual of 18 additional subjects to a Th2 cell treatment arm will provide either 72%, 87%, or 95% power to detect a Th2-mediated reduction in the incidence of grade II to IV GVHD from 60% to 20%. Specifically, the initial incidence, from the phase I accrual of grade II to IV acute GVHD for the Th2 cell dose selected for the phase II component will be either 2/6, 1/6, or 0/6. For these conditions, the statistical power for detecting a reduction in grade II to IV GVHD from 60% to 20% would be 72%, 87%, or 95%, respectively.

To help ensure that the Th2 cells continue to be safely administered in the expanded cohort, the same accrual and stopping rules pertai ing to severe toxicity attributed to the Th2 cells will be continued. Specifically, 24 total patients (6 in the Phase I cohort, 18 in the expanded Phase II cohort) will be evaluated at either Th2 cell dose level #2 or level #3. Accrual and stopping rules pertaining to severe toxicity attributable to Th2 cells will be applied after each cohort of six patients. Therefore, if at any point, the frequency of severe toxicity attributable to the Th2 cells exceeds 1/6, 2/12, 3/18, or 4/24, then accrual to that treatment arm will be stopped.

An additional accrual and stopping rule pertaining to acute GVHD will be utilized in the expanded Phase II cohort. The incidence of grade II to IV acute GVHD in non-Th2 recipients was 12/19, or 63%. In the expanded cohort of Th2 recipients, the incidence of grade II to IV acute GVHD will be calculated on an ongoing basis and reviewed weekly. If at any point in protocol implementation the incidence of grade II to IV acute GVHD in Th2 recipients is 60% or greater, then further accrual to the protocol will be stopped. Up to 2/6 cases of grade II to IV acute GVHD will be allowed for expansion of Th2 accrual to the phase II component Therefore, it is possible that the phase II component of the Th2 accrual may be stopped after 4 patients (if each develops grade II to IV acute GVHD).

In summary, the results disclosed herein indicate that immunoablative reduced intensity allogeneic PBSCT with donor Th2 cells (such as the 25×10⁶/kg dose) is associated with rapid engraftment, GVL effects, and a favorable GVHD incidence and severity relative to conventional allogeneic transplantation using single-agent CSA GVHD prophylaxis.

### EXAMPLE 14

### Th2 cells in the Treatment of Autoimmune Disorders

Allogeneic and autologous Th2 cell transplantation can be used for treatment of autoimmune disorders. Subjects who could benefit from this form of therapy are individuals with severe autoimmunity that is not responsive to conventional treatment approaches. Such subjects can include, but are not limited to, those with rheumatoid arthritis, Chron's disease, systemic lupus erythemetous, or multiple sclerosis.

With the use of allogeneic Th2 cells to treat autoimmune disorders, the recipient's immune system, which is initiating the autoimmune syndrome, will be replaced by a healthy allogeneic donor immune system-L In this treatment protocol, the recipient's immune system is depleted, for example by using chemotherapy as described in EXAMPLE 8. In one embodiment, chemotherapy includes fludarabine in combination with other chemotherapy agents that synergistically induce immune depletion (e.g. agents found in the EPOCH chemotherapy regimen).

Once immune depletion has occurred, the recipient receives preparative regimen chemotherapy with fludarabine and cyclophosphamide, followed by transplantation of a T cell-replete allogeneic peripheral blood stem cell product. This stem cell product is collected after G-CSF mobilization, and contains at least 4×10⁶ donor CD34⁺ stem cells/kg and from 40 to 400×10⁶ donor immune T cells/kg (containing both CD4⁺ and CD8⁺ subsets). On the day following PBSCT, ex vivo generated donor CD4^{'} Th2 cells are administered at a therapeutically effective dose, such as a dose range of 25 to 125×10⁶ cells/kg. With this approach, it is likely that 100% donor lymphoid and myeloid engraftment will occur in such autoimmune recipients. Reconstitution of immunity with a normal donor immune system then alleviates the autoimmune disease.

When autologous Th2 cells are utilized to treat autoimmune disease, recipients are treated with immune-depleting chemotherapy to eliminate the B and T cell populations that contribute to the autoimmune disease. In one embodiment, the chemotherapy includes administration of fludarabine in combination with other chemotherapeutic agents (e.g. drugs found in the EPOCH chemotherapy regimen). The recipient is then additionally treated with a more intensive immune-depleting regimen containing fludarabine with a higher dose of cyclophosphamide. After immune depletion, the recipient is reconstituted with autologous peripheral blood cells that contain both CD4⁺ and CD8⁺ immune T cells. Within 24 hours after such transplantation, autologous CD4⁺ Th2 cells are administered in the dose range of 25 to 125×10⁶ cells/kg. With this approach, based on the clinical results described in EXAMPLE 12, the autologous immune system is reconstituted with a shift towards a more anti-inflammatory, Th2-type immune profile. This Th2-driven immune reconstitution will result in a therapeutic effect, such as a reduction of symptoms of the autoimmune disease, or a decrease in tissue destruction of the target of the autoimmune disease.

### EXAMPLE 15

### Th2 Cells in Solid Organ Transplantation

Graft rejection remains a serious obstacle for the use of solid organ transplantation to treat end-organ failure. Host immune cells that recognize donor alloantigens present on the solid organ graft are responsible for graft rejection. In allogeneic stem cell transplantation involving Th2 cells, 100% donor lymphoid engraftment occurs without a high rate of GVHD. Alloengraftment with reduced GVHD after Th2 infusion also represents an opportunity to transplant donor-type solid organ grafts without graft rejection.

In this method, subjects having end-organ failure are immuno-depleted, for example using the immune-depleting chemotherapy described in EXAMPLE 8. Subjects eligible for this approach include, but are not limited to, those with lung failure, renal failure, heart failure, liver failure, pancreatic islet cell failure, and those with resultant diabetes mellitus. In one example, chemotherapy consists of fludarabine in combination with the chemotherapy agents found in the EPOCH regimen described above. Subsequently, recipients are treated with fludarabine combined with higher doses of cyclophosphamide. After this immune-depleting chemotherapy, recipients receive a T cell-replete allogeneic peripheral blood stem cell transplantation from the individual who will donate the solid organ graft, or from an individual HLA-matched with the solid organ donor. Within about 24 hours of receiving the stem cell transplant, subjects then receive additional donor CD4⁺ Th2 cells, such as between 25 and 125×10⁶ cells/kg. Once complete lymphoid alloengraftment is achieved with this Th2 approach, the recipient then receives the solid organ transplant from the donor.

### EXAMPLE 16

### Pharmaceutical Compositions and Modes of Administration

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Methods of introduction include, but are not limited to, intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal, vaginal and intestinal mucosa, etc.) and may be administered together with other biologically active agents. Administration can be systemic or local. Pharmaceutical compositions can be introduced into the central nervous system by any suitable route, including intraventricular and intrathecal injection; intraventricular injection may be facilitated by an intraventricular catheter, for example, attached to a reservoir, such as an Ommaya reservoir.

The present disclosure also provides pharmaceutical compositions which include a therapeutically effective amount of purified Th2 cells, alone or with a pharmaceutically acceptable carrier. Furthermore, the pharmaceutical compositions or methods of treatment can be administered in combination with other therapeutic treatments, such as chemotherapeutic agents and/or anti-tumor therapies.

Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the purified Th2 cells herein disclosed. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (e.g., powder, pill, tablet, or capsule forms), conventional non-toxic solid carriers can include, for example, pharmaceutical grades of mannitol, lactose, starch, sodium saccharide, cellulose, magnesium carbonate, or magnesium stearate. The composition can be formulated as a suppository, with traditional binders and carriers such as triglycerides.

Embodiments of the disclosure comprising medicaments can be prepared with conventional pharmaceutically acceptable carriers, adjuvants and counterions as would be known to those of skill in the art.

The amount of purified Th2 cells effective in the treatment of a particular disorder or condition will depend on the nature of the disorder or condition, and can be determined by standard clinical techniques. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration Instructions for use of the composition can also be included.

Such compositions are useful as therapeutic agents when constituted as pharmaceutical compositions with the appropriate carriers or diluents.

In view of the many possible embodiments to which the principles of our disclosure may be applied, it should be recognized that the illustrated embodiments are only particular examples of the disclosure and should not be taken as a limitation on the scope of the disclosure. Rather, the scope of the disclosure is in accord with the following claims. We therefore claim all that comes within the scope and spirit of these claims.

We claim:

1. A method of producing a population of substantially purified CD4⁺ Th2 lymphocytes, comprising:
stimulating a population of substantially purified CD4⁺ T cells isolated from a subject by contacting the population with anti-CD3 monoclonal antibody and antibody that specifically binds to a T cell costimulatory molecule, in the presence of a Th2 supportive environment, thereby producing a population of substantially purified CD4⁺ Th2 lymphocytes which secrete at least one Th2 cytokine.

2. The method of claim 1, wherein the Th2 supportive environment comprises at least 200 IU/ml of IL-4.

3. The method of claim 2, wherein the Th2 supportive environment comprises at least 500 IU/ml of IL-4.

4. The method of claim 3, wherein the Th2 supportive environment comprises about 1000 IU/ml of IL-4.

5. The method of claim 2, wherein the Th2 supportive environment further comprises no more than about 10 IU/ml of IL-2.

6. The method of claim 2, wherein the Th2 supportive environment further comprises no more than about 20 IU/ml of IL-2.

7. The method of claim 2 wherein the Th2 supportive environment further comprises less than 5 IU/ml of IL-2.

8. The method of claim 7 wherein the Th2 supportive environments further comprises less than 1 IU/ml of IL-2.

9. The method of claim 2, wherein the Th2 supportive environment further comprises about 0.004 to about 0.1 μM rapamycin.

10. The method of claim 1, further comprising allowing the stimulated population of CD4⁺ T cells to proliferate in the Th2 supportive environment.

11. The method of claim 10, wherein the Th2 supportive environment comprises about 1000 IU/ml of IL-4.

12. The method of claim 11, wherein the Th2 supportive environment further comprises at least about 1 IU/ml of IL-2.

13. The method of claim 11, wherein the Th2 supportive environment further comprises no more than about 20 IU/ml of IL-2.

14. The method of claim 1, wherein the substantially purified CD4⁺ T cells are further purified into a CD4⁺RO⁺ T cell population.

15. The method of claim 1, wherein the at least one Th2 cytokine is IL-4, IL-5 or IL-10.

16. The method of claim 1, wherein the population of substantially purified CD4⁺ Th2 lymphocytes comprises less than 5% Th1 lymphocytes.

17. The method of claim 16, wherein the population of substantially purified CD4⁺ Th2 lymphocytes comprises less than 1% Th1 lymphocytes.

18. The method of claim 1, wherein the population of substantially purified CD4⁺ Th2 lymphocytes produces less than 10 pg/ml of IL-2 per 1×10⁶ CD4⁺ Th2 lymphocytes.

19. The method of claim 1, wherein the population of substantially purified CD4⁺ Th2 lymphocytes produces at least 1000 pg/ml of IL-4 per 1×10⁶ CD4⁺ Th2 lymphocytes.

20. The method of claim 1, further comprising comparing the purity of the population of substantially purified CD4⁺ Th2 lymphocytes with a substantially purified population of purified CD4⁺ Th1 cells.

21. The method of claim 1, further comprising re-stimulating the substantially purified CD4⁺ Th2 lymphocytes with an immobilized anti-CD3 monoclonal antibody and an immobilized antibody that specifically binds to a T cell costimulatory molecule after allowing the cells to proliferate in the Th2 supportive environment.

22. The method of claim 21, wherein the re-stimulation of the T-cells occurs within about eight to about twelve days of the initial stimulation of the T cells.

23. The method of claim 1, further comprising cryo-preserving the purified CD4⁺ Th2 lymphocytes.

24. The method of claim 1, wherein the antibody that specifically binds to a T cell costimulatory receptor specifically binds CD28, inducible costimulatory molecule (ICOS), 4-1BB receptor (CDw137), lymphocyte function-associated antigen-1(LFA-1), CD30, or CD154.

25. The method of claim 24, wherein the antibody that specifically binds a T cell costimulatory molecule specifically binds CD28.

26. The method of claim 1, wherein the antibodies are immobilized.

27. The method of claim 26, wherein the antibodies are immobilized on a magnetic solid phase surface.

28. A CD4⁺ Th2 cell produced by the method of claim 1.

29. A method of producing a population of substantially purified CD4⁺ Th2 lymphocytes, comprising:
obtaining a population of CD4⁺ T lymphocytes from a subject;
purifying a population of CD4⁺RO⁺ T cells from the CD4⁺ T lymphocytes;
initially stimulating the CD4⁺ T lymphocytes in a media comprising an anti-CD3 monoclonal antibody, an anti-CD28 monoclonal antibody, about 1000 IU/ml of IL-4, wherein the anti-CD3 monoclonal antibody and the anti-CD28 monoclonal antibody are immobilized on a magnetized solid substrate; and
re-stimulating the T lymphocytes in the media, thereby producing a population of substantially purified CD4⁺ Th2 lymphocytes, wherein the population of CD4⁺ Th2 lymphocytes is substantially free of Th1 lymphocytes.

30. The method of claim 29, wherein the media further comprises no more than about 20 IU/ml IL-2.

31. A substantially purified population of CD4⁺ Th2 lymphocytes, wherein the population comprises less than 5% CD4⁺ Th1 lymphocytes.

32. The substantially purified population of CD4⁺ Th2 lymphocytes of claim 31 wherein the population comprises less than 1% CD4⁺ Th1 lymphocytes.

33. The substantially purified population of CD4⁺ Th2 lymphocytes of claim 32, wherein the population produces less than about 200 pg/μg of IL-2 per 1×10⁶ CD4⁺ Th2 lymphocytes.

34. The substantially purified population of CD4⁺ Th2 lymphocytes of claim 33, wherein the population produces less than about 100 pg/ml of IL-2 per 1×10⁶ CD4⁺ Th2 lymphocytes.

35. The substantially purified population of CD4⁺ Th2 lymphocytes of claim 34, wherein the population produces less than about 10 pg/μg of IL-2 per 1×10⁶ CD4⁺ Th2 lymphocytes.

36. The substantially purified population of CD4⁺ Th2 lymphocytes of claim 32, wherein the population produces at least 1000 pg/ml of IL-4 per 1×10⁶ CD4⁺ Th2 lymphocytes.

37. A method of transplanting allogeneic donor immune cells to reconstitute immunity in a recipient having a tumor, comprising:
depleting at least the recipient's T cells that mediate graft rejection;
administering to the recipient a therapeutically effective amount of a population of allogeneic cells comprising CD4⁺ and CD8⁺ T cells; and
administering to the recipient a therapeutically effective amount of a population of CD4⁺ Th2 cells, thereby transplanting allogeneic immune cells into the recipient and reconstituting immunity in the recipient.

38. The method of claim 37, wherein the tumor is a carcinoma.

39. The method of claim 38, wherein the carcinoma is a renal cell carcinoma, ovarian cancer, breast cancer, colon cancer or malignant melanoma.

40. The method of claim 37, wherein the population of donor allogeneic cells comprising CD4⁺ and CD8⁺ T cells are administered as a peripheral blood stem cell product.

41. The method of claim 37, wherein the tumor is acute lymphocytic leukemia, acute mylogenous leukemia, chronic lymphocytic leukemia, chronic myelogenous leukemia, acute myelogenous leukemia, chronic lymphocytic leukemia, chronic myelogenous leukemia, indolent non-Hodgkin's lymphoma, high-grade non-Hodgkin's lymphoma, Hodgkin's lymphoma, multiple myeloma, or myelodysplastic syndrome.

42. The method of claim 37, wherein depleting the recipient's T cells comprises administering to the recipient an induction chemotherapy regimen comprising a therapeutically effective amount of etoposide, doxorubicin, vincristine, cyclophosphamide, and prednisone.

43. The method of claim 42, wherein the induction chemotherapy regimen further comprises administering to the recipient a therapeutically effective amount of fludarabine.

44. The method of claim 37, wherein depleting the recipient's T cells further comprises administering to the recipient a transplant preparative chemotherapy regimen comprising a therapeutically effective amount of fludarabine and cyclophosphamide.

45. The method of claim 37, wherein the allogeneic T cells are from an HLA-matched first degree relative donor.

46. The method of claim 37, wherein the allogeneic peripheral blood cells enriched for CD4⁺ Th2 cells are produced by:
stimulating a population of isolated CD4⁺ T cells by contacting the population with an immobilized anti-CD3 monoclonal antibody and an immobilized antibody that specifically binds a co-stimulatory molecule in the presence of a Th2 supportive environment to form a stimulated population of T cells; and
allowing the stimulated population of T cells to proliferate in a Th2 supportive environment, thereby producing a population of substantially purified donor CD4⁺ Th2 lymphocytes.

47. The method of claim 37, wherein the administration of allogeneic cells comprising CD4⁺ and CD8⁺ T cells and the CD4⁺ Th2 cells is at the same time.

48. The method of claim 37, wherein the CD4⁺ Th2 cells are administered following the administration of the allogeneic cells comprising CD4⁺ and CD8⁺ T cells.

49. The method of claim 37, wherein the administration of the CD4⁺ Th2 cells is within one day of the administration of the allogeneic cells comprising CD4⁺ and CD8⁺ T cells.

50. The method of claim 37, wherein the CD4⁺ Th2 cells are administered at a time remote from the administration of the allogeneic cells comprising CD4⁺ and CD8⁺ T cells.

51. The method of claim 37, wherein the CD4⁺ Th2 cells are administered at a dose of about 5×10⁶ cells per kilogram to about 125×10⁶ cells per kilogram.

52. The method of claim 37, wherein the donor CD4⁺ Th2 cells are administered at a dose of about 25×10⁶ cells per kilogram.

53. A method of treating a subject having an autoimmune disorder, comprising:
depleting at least the subject's T cells that mediate the autoimmune disorder;
administering to the subject a therapeutically effective amount of autologous peripheral blood cells comprising CD4⁺ and CD8⁺ T cells; and
administering to the subject a therapeutically effective amount of autologous CD4⁺ Th2 cells, wherein the administration of the autologous peripheral blood cells and autologous CD4⁺ Th2 cells treats the autoimmune disorder.

54. The method of claim 53 wherein the autoimmune disorder is rheumatoid arthritis, Crohn's disease, systemic lupus erythemetous, multiple sclerosis, or diabetes.

55. A method of preventing or limiting rejection of a solid organ in a recipient, comprising:
depleting at least the recipient's T cells that mediate graft rejection;
administering to the recipient a therapeutically effective amount of allogeneic peripheral blood cells comprising stem cells, CD4⁺ cells, and CD8⁺ cells;
administering to the recipient a therapeutically effective amount of CD4⁺ Th2 cells; and
transplanting a solid organ into the recipient, wherein the solid organ is HLA-matched to the CD4⁺ Th2 cells and the allogeneic peripheral blood cells, wherein administration of the allogeneic peripheral blood cells and the allogeneic CD4⁺ Th2 results in preventing or limiting rejection of the solid organ.

56. The method of claim 55, wherein the organ is a kidney, liver, heart, lung, or pancreas.

57. The method of claim 55, wherein the recipient has a disorder selected from the group consisting of renal failure, kidney failure, heart failure, liver failure, lung failure, or diabetes.

58. The method of claim 57, wherein the solid organ, the CD4⁺ Th2 cells and the allogeneic peripheral blood cells are from the same donor.

59. A method of decreasing a graft-versus-host-disease (GVHD) response in a subject, comprising:
administering to the subject a composition comprising a population of substantially purified CD4⁺ Th2 lymphocytes prepared using the method of claim 1, wherein administration of the population of substantially purified CD4⁺ Th2 lymphocytes decreases a GVHD response in the subject.

60. The method of claim 59, wherein the population of substantially purified CD4⁺ Th2 lymphocytes are cryopreserved and thawed prior to administration to the subject.

61. The method of claim 59, wherein the population of substantially purified CD4⁺ Th2 lymphocytes are administered at a dose of about 5×10⁶ to about 2×10⁸ substantially purified CD4⁺ Th2 lymphocytes per kilogram of subject

62. The method of claim 59, wherein the composition further comprises a pharmaceutically acceptable carrier.

63. The method of claim 59, wherein the composition further comprises non-cultured CD4⁺ and CD8⁺ T cells.

64. The method of claim 59, wherein the composition is administered to treat a tumor.

65. The method of claim 64 wherein the tumor is a hematological or solid tumor.

66. The method of claim 59, further comprising administering a chemotherapeutic agent, or a monoclonal antibody, to the subject.

67. The method of claim 65, wherein the solid tumor is a renal cell carcinoma, ovarian cancer, breast cancer, colon cancer or malignant melanoma.

68. The method of claim 65, wherein the hematological tumor is a leukemia; acute lymphocytic leukemia; acute myelocytic leukemia; acute myelogenous leukemia; myeloblastic, promyelocytic, myelomonocytic, monocytic and erythroleukemia; chronic myelocytic (granulocytic) leukemia; chronic myelogenous leukemia; chronic lymphocytic leukemia; polycythemia vera; lymphoma; Hodgkin's disease; non-Hodgkin's lymphoma (indolent and high grade forms); multiple myeloma; Waldenstrdm's macroglobulinemia; heavy chain disease; myelodysplastic syndrome; or a myelodysplasia.

69. A method of treating a subject having at least one tumor comprising:
administering an immuno-depleting agent to the subject; and
administering a population of substantially purified CD4⁺ Th2 lymphocytes prepared using the method of claim 1 to the subject, wherein administration of the substantially purified CD4⁺ Th2 lymphocytes treats the tumor.

70. The method of claim 69, wherein the immuno-depleting agent is a chemotherapeutic agent or monoclonal antibody.

71. The method of claim 69, wherein the population of substantially purified CD4⁺ Th2 lymphocytes are administered at a dose of about 5×10⁶ cells per kilogram to about 125×10⁶ cells per kilogram
